(19) United States
(12) Patent Application Publication
(10) Pub. No.: US 2025/0152942 A1
(43) Pub. Date: May 15, 2025

(54) DETERMINING STIMULATION AND SENSING AREAS FOR ADAPTIVE NEUROSTIMULATION TREATMENTS

(71) Applicant: Boston Scientific Neuromodulation Corporation, Valencia, CA (US)

(72) Inventors: Andrew James Haddock, Los Angeles, CA (US); Tianhe Zhang, Studio City, CA (US); Jonathan Philip Larcom, Simi Valley, CA (US)

(21) Appl. No.: 18/937,202

(22) Filed: Nov. 5, 2024

Related U.S. Application Data
(60) Provisional application No. 63/597,810, filed on Nov. 10, 2023.

(51) Int. Cl. A61N 1/36; A61N 1/372

(52) U.S. Cl. CPC A61N 1/36139; A61N 1/36146; A61N 1/37247

(57) ABSTRACT

Systems and techniques are disclosed for determining and implementing neurostimulation programming, based on sensing and therapy capabilities. An example technique includes: identifying an arrangement for a plurality of electrical contacts of one or more implanted leads; identifying neurostimulation parameters for delivering therapy to a neurostimulation location using a first set of electrical contacts of the arrangement; determining a sensing capability for sensing an evoked response to the therapy delivered to the neurostimulation location, using a second set of electrical contacts of the arrangement, and determining the sensing capability based on the arrangement and the neurostimulation parameters; and changing at least a portion of the neurostimulation parameters for delivering the therapy, in response to a determination that the sensing capability is not available at the neurostimulation location. The changed neurostimulation parameters can be programmed and used for delivering therapy to a changed neurostimulation location that provides suitable sensing capabilities.

### CLAIM OF PRIORITY

This application claims the benefit of U.S. Provisional Application No. 63/597,810, filed on Nov. 10, 2023, which is hereby incorporated by reference in its entirety.

### TECHNICAL FIELD

The present disclosure relates generally to data processing in connection with the use of medical devices, and more particularly, to systems, devices, and methods for managing neurostimulation therapy and sensing capabilities, including with implanted electrical stimulation devices and neurostimulation programming in such devices to treat pain, movement disorders, and/or related physiological conditions.

### BACKGROUND

Neurostimulation, also referred to as neuromodulation, has been proposed as a therapy for a number of conditions. Examples of neuromodulation include Spinal Cord Stimulation (SCS), Deep Brain Stimulation (DBS), Peripheral Nerve Stimulation (PNS), and Functional Electrical Stimulation (FES). Implantable neuromodulation systems have been applied to deliver such a therapy. An implantable neuromodulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system.

A neuromodulation system can be used to electrically stimulate tissue or nerve centers to treat nervous or muscular disorders. For example, an SCS system may be configured to deliver electrical pulses to a specified region of a patient's spinal cord, such as particular spinal nerve roots or nerve bundles, to produce an analgesic effect that masks pain sensation, or to produce a functional effect that allows increased movement or activity of the patient. Other forms of neurostimulation may include a DBS system that uses similar pulses of electricity at particular locations in the brain to reduce symptoms of essential tremor, Parkinson's disease, epilepsy, psychological disorders, or the like.

To enable an improved treatment of a particular patient using a neuromodulation system, sensing data (e.g., captured via the one or more electrodes) may be analyzed to measure the effectiveness of therapy waveforms, to drive improvements to system programming. These types of systems are generally referred to as “adaptive” neuromodulation systems. Adaptive neuromodulation uses both stimulation (therapy from electrode outputs) and sensing (measurements from electrode inputs) determine programming that is compatible with therapy delivery and electrophysiological recording goals. However, these two goals can sometimes be at odds, because therapy delivery may interfere with some sensing capabilities (even though therapy delivery may be a primary objective of programming).

### SUMMARY

The following Summary provides examples as an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

Example 1 is a system, comprising: one or more processors; and one or more memory devices comprising instructions, which when executed by the one or more processors, cause the one or more processors to perform operations that: identify an arrangement for a plurality of electrical contacts of one or more implanted leads; identify neurostimulation parameters for delivering therapy to a neurostimulation location using a first set of electrical contacts of the arrangement; determine a sensing capability for sensing an evoked response to the therapy delivered to the neurostimulation location, using a second set of electrical contacts of the arrangement, wherein the sensing capability is determined based on the arrangement and one or more of the neurostimulation parameters; and change at least a portion of the neurostimulation parameters for delivering the therapy, in response to a determination that the sensing capability is not available at the neurostimulation location, to provide changed neurostimulation parameters for delivering therapy to a changed neurostimulation location.

In Example 2, the subject matter of Example 1 optionally includes subject matter where the neurostimulation location corresponds to a central point of stimulation provided with the first set of electrical contacts.

In Example 3, the subject matter of any one or more of Examples 1-2 optionally include subject matter where to determine the sensing capability at the neurostimulation location includes to determine whether an evoked compound action potential (ECAP) is observable.

In Example 4, the subject matter of any one or more of Examples 1-3 optionally include subject matter where the operations to identify the neurostimulation parameters, to determine the sensing capability, and to change the neurostimulation parameters, are iteratively repeated to produce the changed neurostimulation parameters associated with changed neurostimulation locations, until an evoked compound action potential (ECAP) is observed from use of the changed neurostimulation parameters at one of the changed neurostimulation locations.

In Example 5, the subject matter of any one or more of Examples 1-4 optionally include subject matter where the instructions further cause the one or more processors to perform operations that: determine that the sensing capability is available at the changed neurostimulation location; and enable use of the changed neurostimulation parameters within one or more programs of a neurostimulation device.

In Example 6, the subject matter of Example 5 optionally includes subject matter where the instructions further cause the one or more processors to perform operations that: receive an indication that the therapy remains effective for a patient at the changed neurostimulation location; wherein the changed neurostimulation parameters are enabled for use within the one or more programs in response to the indication that the therapy remains effective for the patient.

In Example 7, the subject matter of Example 6 optionally includes subject matter where the instructions further cause the one or more processors to perform operations that: determine that the sensing capability is available via the second set of electrical contacts of the arrangement, based on use of the changed neurostimulation parameters; wherein the changed neurostimulation parameters are enabled for use within the one or more programs in response to a determination that both the sensing capability remains available and the therapy remains effective for the patient.

In Example 8, the subject matter of any one or more of Examples 1-7 optionally include subject matter where the change to the neurostimulation parameters is based on a change of a center point of stimulation relative to a suggested sensing area, and wherein a size and a location of the suggested sensing area is based on lead geometry and stimulation waveform characteristics.

In Example 9, the subject matter of any one or more of Examples 1-8 optionally include subject matter where the change to the neurostimulation parameters is based on delivering therapy to the changed neurostimulation location outside of a sensing exclusion area, and wherein a size and a location of the sensing exclusion area is based on lead geometry and stimulation waveform characteristics.

In Example 10, the subject matter of any one or more of Examples 1-9 optionally include subject matter where the instructions further cause the one or more processors to perform operations that: display, in a programming user interface, a representation of the neurostimulation location and a representation of the plurality of electrical contacts; wherein the change to the neurostimulation parameters is based on settings of the neurostimulation parameters that are modifiable in the programming user interface.

In Example 11, the subject matter of Example 10 optionally includes subject matter where the instructions further cause the one or more processors to perform operations that: display, in the programming user interface, a representation of a suggested sensing area, relative to the area of the therapy; and receive user interaction in the programming user interface to change at least one value of the neurostimulation parameters or the neurostimulation location, based on the suggested sensing area.

In Example 12, the subject matter of any one or more of Examples 10-11 optionally include subject matter where the instructions further cause the one or more processors to perform operations that: display, in the programming user interface, a representation of a sensing exclusion area, relative to the area of the therapy; and receive user interaction in the programming user interface to change at least one value of the neurostimulation parameters or the neurostimulation location, based on the sensing exclusion area.

In Example 13, the subject matter of any one or more of Examples 11-12 optionally include subject matter where the user interaction to change the at least one value of the neurostimulation parameters relates to a change to amplitude, pulse width, frequency, and percentage, of modulated energy to be provided with a particular contact of the plurality of electrical contacts.

Example 14 is a machine-readable medium including instructions, which when executed by a machine, cause the machine to perform the operations of the system of any of the Examples 1 to 13.

Example 15 is a method to perform the operations of the system of any of the Examples 1 to 13.

Example 16 is a device to identify therapy and sensing capabilities of neurostimulation programming, the device comprising: one or more processors; and one or more memory devices comprising instructions, which when executed by the one or more processors, cause the one or more processors to: identify an arrangement for a plurality of electrical contacts of one or more implanted leads; identify neurostimulation parameters for delivering therapy to a neurostimulation location using a first set of electrical contacts of the arrangement; determine a sensing capability for sensing an evoked response to the therapy delivered to the neurostimulation location, using a second set of electrical contacts of the arrangement, wherein the sensing capability is determined based on the arrangement and one or more of the neurostimulation parameters; and change at least a portion of the neurostimulation parameters for delivering the therapy, in response to a determination that the sensing capability is not available at the neurostimulation location, to provide changed neurostimulation parameters for delivering therapy to a changed neurostimulation location.

In Example 17, the subject matter of Example 16 optionally includes subject matter where the neurostimulation location corresponds to a central point of stimulation provided with the first set of electrical contacts, and wherein to determine the sensing capability at the neurostimulation location includes to determine whether an evoked compound action potential (ECAP) is observable.

In Example 18, the subject matter of any one or more of Examples 16-17 optionally include subject matter where operations to identify the neurostimulation parameters, to determine the sensing capability, and to change the neurostimulation parameters, are iteratively repeated to produce the changed neurostimulation parameters associated with changed neurostimulation locations, until an evoked compound action potential (ECAP) is observed from use of the changed neurostimulation parameters at one of the changed neurostimulation locations.

In Example 19, the subject matter of any one or more of Examples 16-18 optionally include subject matter where the instructions further cause the one or more processors to perform operations that: determine that the sensing capability is available at the changed neurostimulation location; and enable use of the changed neurostimulation parameters within one or more programs of a neurostimulation device.

In Example 20, the subject matter of Example 19 optionally includes subject matter where the instructions further cause the one or more processors to perform operations that: receive an indication that the therapy remains effective for a patient at the changed neurostimulation location; wherein the changed neurostimulation parameters are enabled for use within the one or more programs in response to the indication that the therapy remains effective for the patient.

In Example 21, the subject matter of Example 20 optionally includes subject matter where the instructions further cause the one or more processors to perform operations that: determine that the sensing capability is available via the second set of electrical contacts of the arrangement, based on use of the changed neurostimulation parameters; wherein the changed neurostimulation parameters are enabled for use within the one or more programs in response to a determination that both the sensing capability remains available and the therapy remains effective for the patient.

In Example 22, the subject matter of Example 21 optionally includes subject matter where the change to the neurostimulation parameters is based on a suggested sensing area or a sensing exclusion area; wherein a size and a location of the suggested sensing area or the sensing exclusion area is based on lead geometry and stimulation waveform characteristics.

In Example 23, the subject matter of any one or more of Examples 16-22 optionally include subject matter where the instructions further cause the one or more processors to: display, in a programming user interface, a representation of the neurostimulation location and a representation of the plurality of electrical contacts; wherein the change to the neurostimulation parameters is based on settings of the neurostimulation parameters that are modifiable in the programming user interface.

In Example 24, the subject matter of Example 23 optionally includes subject matter where the instructions further cause the one or more processors to: display, in the programming user interface, a representation of a suggested sensing area, relative to the area of the therapy; display, in the programming user interface, a representation of a sensing exclusion area, relative to the area of the therapy; and receive user interaction in the programming user interface to change at least one value of the neurostimulation parameters or the neurostimulation location, based on the suggested sensing area or the sensing exclusion area.

In Example 25, the subject matter of Example 24 optionally includes subject matter where the user interaction to change the at least one value of the neurostimulation parameters relates to a change to amplitude, pulse width, frequency, and percentage, of modulated energy to be provided with a particular contact of the plurality of electrical contacts.

Example 26 is a method for identifying therapy and sensing capabilities of neurostimulation programming, comprising: identifying an arrangement for a plurality of electrical contacts of one or more implanted leads; identifying neurostimulation parameters for delivering therapy to a neurostimulation location using a first set of electrical contacts of the arrangement; determining a sensing capability for sensing an evoked response to the therapy delivered to the neurostimulation location, using a second set of electrical contacts of the arrangement, wherein the sensing capability is determined based on the arrangement and one or more of the neurostimulation parameters; and changing at least a portion of the neurostimulation parameters for delivering the therapy, in response to a determination that the sensing capability is not available at the neurostimulation location, to provide changed neurostimulation parameters for delivering therapy to a changed neurostimulation location.

In Example 27, the subject matter of Example 26 optionally includes subject matter where the neurostimulation location corresponds to a central point of stimulation provided with the first set of electrical contacts, and wherein determining the sensing capability at the neurostimulation location includes determining whether an evoked compound action potential (ECAP) is observable.

In Example 28, the subject matter of any one or more of Examples 26-27 optionally include subject matter where identifying the neurostimulation parameters, determining the sensing capability, and changing the neurostimulation parameters, are iteratively repeated to produce the changed neurostimulation parameters associated with changed neurostimulation locations, until an evoked compound action potential (ECAP) is observed from use of the changed neurostimulation parameters at one of the changed neurostimulation locations.

In Example 29, the subject matter of any one or more of Examples 26-28 optionally include determining that the sensing capability is available at the changed neurostimulation location; and enabling use of the changed neurostimulation parameters within one or more programs of a neurostimulation device.

In Example 30, the subject matter of Example 29 optionally includes receiving an indication that the therapy remains effective for a patient at the changed neurostimulation location; wherein the changed neurostimulation parameters are enabled for use within the one or more programs in response to the indication that the therapy remains effective for the patient.

In Example 31, the subject matter of Example 30 optionally includes determining that the sensing capability is available via the second set of electrical contacts of the arrangement, based on use of the changed neurostimulation parameters; wherein the changed neurostimulation parameters are enabled for use within the one or more programs in response to determining that both the sensing capability remains available and the therapy remains effective for the patient.

In Example 32, the subject matter of Example 31 optionally includes subject matter where the change to the neurostimulation parameters is based on a suggested sensing area or a sensing exclusion area; wherein a size and a location of the suggested sensing area or the sensing exclusion area is based on lead geometry and stimulation waveform characteristics.

In Example 33, the subject matter of any one or more of Examples 26-32 optionally include displaying, in a programming user interface, a representation of the neurostimulation location and a representation of the plurality of electrical contacts; wherein the change to the neurostimulation parameters is based on settings of the neurostimulation parameters that are modifiable in the programming user interface.

In Example 34, the subject matter of Example 33 optionally includes displaying, in the programming user interface, a representation of a suggested sensing area, relative to the area of the therapy; displaying, in the programming user interface, a representation of a sensing exclusion area, relative to the area of the therapy; and receive user interaction in the programming user interface to change at least one value of the neurostimulation parameters or the neurostimulation location, based on the suggested sensing area or the sensing exclusion area.

In Example 35, the subject matter of Example 34 optionally includes subject matter where the user interaction to change the at least one value of the neurostimulation parameters relates to a change to amplitude, pulse width, frequency, and percentage, of modulated energy to be provided with a particular contact of the plurality of electrical contacts.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example, an embodiment of a neurostimulation system.

FIG. 2 illustrates, by way of example, an embodiment of a stimulation device and a lead system, such as may be implemented in the neurostimulation system of FIG. 1.

FIG. 3 illustrates, by way of example, an embodiment of a programming device, such as may be implemented in the neurostimulation system of FIG. 1.

FIG. 4 illustrates, by way of example, an implantable neurostimulation system and portions of an environment in which the system may be used.

FIG. 5 illustrates, by way of example, an embodiment of an implantable stimulator and one or more leads of a neurostimulation system, such as the implantable neurostimulation system of FIG. 4.

FIG. 6 illustrates, by way of example, an embodiment of a programming system and patient data analysis system for use with a neurostimulation system, such as the implantable neurostimulation system of FIG. 4.

FIG. 7 illustrates, by way of example, a user interface operable in connection with user control of therapy and sensing objectives for neurostimulation treatments.

FIGS. 8A, 8B, and 8C illustrate, by way of example, user interfaces to enable sensing functionality used for neurostimulation programming.

FIG. 9 illustrates, by way of example, a flowchart of an example process for automated sensing reconciliation.

FIGS. 10A, 10B, 10C, 10D, and 10E illustrate, by way of example, user interfaces to enable further sensing functionality used for neurostimulation programming.

FIG. 11 illustrates, by way of example, a data graph showing a relationship between stimulation and sensing objectives.

FIG. 12 illustrates, by way of example, an adaptive data processing flow for neurostimulation treatment incorporating therapy and sensing programming objectives.

FIG. 13 illustrates, by way of example, a flowchart of a method implemented by a system or device to identify therapy and sensing capabilities in connection with neurostimulation programming.

FIG. 14 illustrates, by way of example, a block diagram of an embodiment of a computing system for performing patient data analysis in connection with the sensing and therapy objectives discussed herein.

FIG. 15 illustrates, by way of example, a block diagram of an embodiment of a computing system implementing neurostimulation programming circuitry, to cause programming of an implantable electrical neurostimulation device.

FIG. 16 is a block diagram illustrating a machine in the example form of a computer system, within which a set or sequence of instructions may be executed to cause the machine to perform any one of the methodologies discussed herein, according to an example embodiment.

### DETAILED DESCRIPTION

This document discusses various techniques for the collection, processing, analysis, and use of therapy and sensing data provided in an adaptive neuromodulation treatment performed by an implantable electrical neurostimulation device. In various examples, approaches are described to obtain user input related to stimulation therapy and sensing objectives. This user input may be analyzed to assist a user in achieving a suitable balance between the therapy objectives (e.g., to deliver pain relief) and sensing objectives (e.g., to capture response data associated with pain relief) of neurostimulation programming. The objectives may be defined to provide a priority of therapy over sensing, or sensing over therapy, or some balance between these objectives.

The techniques discussed herein may be used to reconcile therapy and sensing operations, and may have applicability to different stages of a neuromodulation therapy progression. In a first example use case, the techniques may be used to assist with operating room lead placement, to ensure that the electrodes are implanted and/or operational in an anatomical location that can serve both therapy and sensing functions. In a second example use case, the techniques may be used as part of temporary (test) or permanent programming sessions, where therapy and sensing functions are established for a user via parameter and program adjustments (e.g., via a clinician programming software application). In a third example use case, the techniques may be used as part of ongoing treatment (e.g., in an at-home setting) where a patient is guided to provide programming changes or adjustments (e.g., via a patient- or caregiver-operated smartphone application) that affect the amount or type of sensing data that can be collected in response to a neurostimulation treatment.

The following provides an introduction to the features of an example neurostimulation system and how a neurostimulation system may be programmed to cause specific neurostimulation effects on a subject patient. Thereafter, the following discusses how stimulation therapy and sensing objectives may be evaluated for a particular patient during programming, including with the identification of suggested sensing areas, exclusion areas, and various adaptations of automated or manually guided therapies. Finally, various examples are provided to identify the effects of particular programming settings on sensing capabilities and functions, such as effects based on neuromodulation waveform shapes and conditions (including effects on sensing caused by pulse width and frequency variations). While neurostimulation therapies, such as SCS and DBS therapies, are specifically discussed as examples, the present subject matter may apply to other therapies that employ stimulation pulses of electrical or other forms of energy for treating chronic pain or similar physiological or medical conditions.

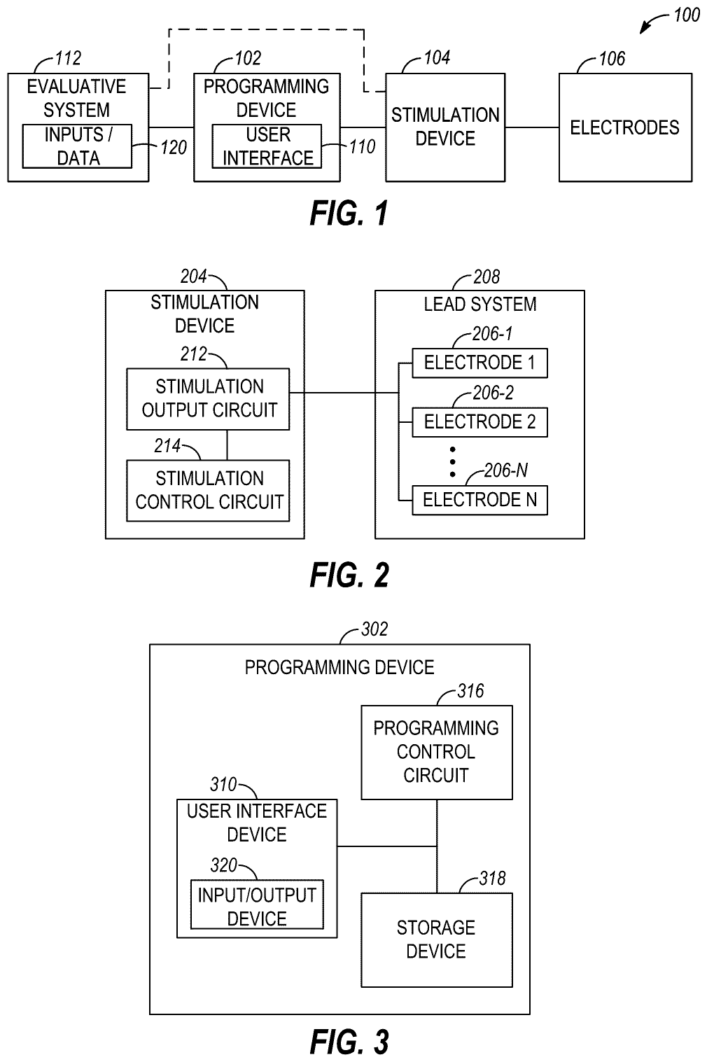

FIG. 1 illustrates an embodiment of a neurostimulation system 100. System 100 includes electrodes 106, a stimulation device 104, and a programming device 102. Electrodes 106 are configured to be placed on or near one or more neural targets in a patient. Stimulation device 104 is configured to be electrically connected to electrodes 106 and deliver neurostimulation energy, such as in the form of electrical pulses, to the one or more neural targets though electrodes 106. The delivery of the neurostimulation is controlled by using a plurality of stimulation parameters, such as stimulation parameters specifying a pattern of the electrical pulses and a selection of electrodes through which each of the electrical pulses is delivered. In various embodiments, at least some parameters of the plurality of stimulation parameters are selected or programmable by a clinical user, such as a physician or other caregiver who treats the patient using system 100; however, some of the parameters may also be provided in connection with automated (e.g., closed-loop or partially-closed-loop) programming logic and adjustment, including the adaptive approaches that balance sensing and therapy objectives and functions as discussed herein. Programming device 102 provides the user with accessibility to implement, change, or modify the programmable parameters. In various embodiments, programming device 102 is configured to be communicatively coupled to stimulation device 104 via a wired or wireless link.

In various embodiments, programming device 102 includes a user interface 110 (e.g., a user interface embodied by a graphical, text, voice, or hardware-based user interface) that allows the user to set and/or adjust values of the user-programmable parameters by creating, editing, loading, and removing programs that include parameter combinations such as patterns and waveforms. Such waveforms may include, for example, the waveform of a pattern of neurostimulation pulses to be delivered to the patient as well as individual waveforms that are used as building blocks of the pattern of neurostimulation pulses. Examples of such individual waveforms include pulses, pulse groups, and groups of pulse groups. The program and respective sets of parameters may also define an electrode selection specific to each individually defined waveform.

The neurostimulation energy that is discussed herein may be delivered in the form of electrical neurostimulation pulses. The delivery is controlled using stimulation parameters that specify spatial (where to stimulate), temporal (when to stimulate), and informational (patterns of pulses directing the nervous system to respond as desired) aspects of a pattern of neurostimulation pulses. Many current neurostimulation systems are programmed to deliver periodic pulses with one or a few uniform waveforms continuously or in bursts. However, neural signals may include more sophisticated patterns to communicate various types of information, including sensations of pain, pressure, temperature, etc.

The present approaches further provide examples of an evaluative system 112, such as a patient data analysis system, which is used to analyze data related to sensing and therapy objectives based on patient data or inputs 120 related to a prior, ongoing, or planned neurostimulation treatment. This evaluative system 112 can initiate a data processing action related to programming and projected effects of the neurostimulation treatment based on analysis of patient data or inputs 120 including sensing data measurements (e.g., detection of an evoked compound action potential (ECAP) or another measurement of neural activity observed at a particular location). In further examples, the patient data or inputs 120 may include feedback or instructions collected from the patient, a clinician or other medical provider, a caregiver, another third party, and directly include data provided from sensors and data monitoring devices to be analyzed by the evaluative system 112. The evaluative system 112 may reside at a remote computing system, such as a cloud server that provides services to perform data processing on demand when invoked by the user interface 110 or other entities. In various examples, in addition to reprogramming actions based on adaptive therapy and sensing objectives, the evaluative system 112 may also directly or indirectly collect information regarding the patient or the ongoing neurostimulation treatment by being communicatively coupled with the programming device 102 or the stimulation device 104.

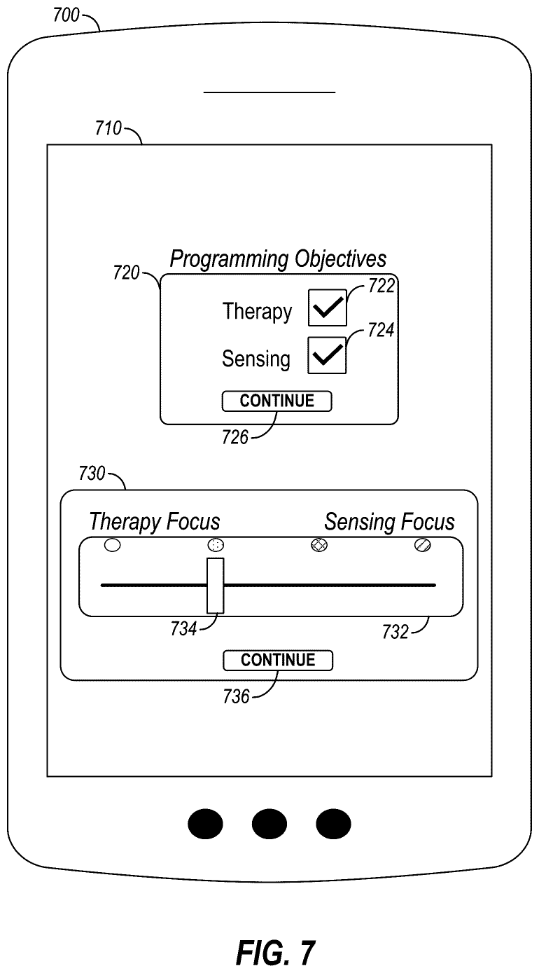

As described in more detail below with respect to the data flows in FIGS. 7 to 13, the evaluative system 112 may evaluate various spatial and localized effects of therapy energy to determine the effects on sensing capabilities by the stimulation device 104. The evaluative system 112 may perform one or more actions to identify, model, classify, forecast, or predict sensing capabilities during the use of particular therapy waveforms, by using artificial intelligence and other algorithmic processing (including rules, data value lookups, etc.). The evaluative system 112 may initiate or control workflows that prompt and measure whether sensing capabilities have been reduced, improved, removed, or added, and provide remedial programming adjustments to a particular waveform or a programming parameter on the stimulation device 104 based on the balancing of therapy and sensing objectives.

Example parameters that can be implemented by a selected neurostimulation program include, but are not limited to the following: amplitude, pulse width, frequency, duration, total charge injected per unit time, cycling (e.g., on/off time), pulse shape, number of phases, phase order, interphase time, charge balance, ramping, as well as spatial variance (e.g., electrode configuration changes over time). As detailed in FIG. 6, a controller, e.g., controller 630 of FIG. 6, can implement program(s) and parameter setting(s) to affect a specific neurostimulation waveform, pattern, or energy output, using a program or setting in storage, e.g., external storage device 616 of FIG. 6, or using settings communicated via an external communication device 618 of FIG. 6 corresponding to the selected program. The implementation of such program(s) or setting(s) may further define a therapy strength and treatment type corresponding to a specific pulse group, or a specific group of pulse groups, based on the specific programs or settings. The evaluative system 112 and its evaluation of the patient data or inputs 120 provides a mechanism to improve the efficacy of treatment and programming settings for a particular patient, and to more effectively capture sensing data that can be used to improve future treatment and programming (in an indirect or direct manner).

Portions of the evaluative system 112, the stimulation device 104 (e.g., implantable medical device), or the programming device 102 can be implemented using hardware, software, or any combination of hardware and software. Portions of the stimulation device 104 or the programming device 102 may be implemented using an application-specific circuit that can be constructed or configured to perform one or more particular functions, or can be implemented using a general-purpose circuit that can be programmed or otherwise configured to perform one or more particular functions. Such a general-purpose circuit can include a microprocessor or a portion thereof, a microcontroller or a portion thereof, or a programmable logic circuit, or a portion thereof. The system 100 could also include a subcutaneous medical device (e.g., subcutaneous ICD, subcutaneous diagnostic device), wearable medical devices (e.g., patch-based sensing device), or other external medical devices.

FIG. 2 illustrates an embodiment of a stimulation device 204 and a lead system with one or more leads 208, such as may be implemented in neurostimulation system 100 of FIG. 1. Stimulation device 204 represents an embodiment of stimulation device 104 and includes a stimulation output circuit 212 and a stimulation control circuit 214. Stimulation output circuit 212 produces and delivers neurostimulation pulses, including the neurostimulation waveform and parameter settings implemented via a program selected or implemented with the user interface 110. Stimulation control circuit 214 controls the delivery of the neurostimulation pulses using the plurality of stimulation parameters, which specifies a pattern of the neurostimulation pulses. Lead system includes one or more leads 208 each configured to be electrically connected to stimulation device 204 and a plurality of electrodes 206 distributed in the one or more leads. The plurality of electrodes 206 includes electrode 206-1, electrode 206-2, . . . electrode 206-N, each a single electrically conductive contact providing for an electrical interface between stimulation output circuit 212 and tissue of the patient, where N≥2. The neurostimulation pulses are each delivered from stimulation output circuit 212 through a set of electrodes selected from electrodes 206. In various embodiments, the neurostimulation pulses may include one or more individually defined pulses, and the set of electrodes may be individually definable by the user for each of the individually defined pulses.

In various embodiments, the number of leads and the number of electrodes on each lead depend on, for example, the distribution of target(s) of the neurostimulation and the need for controlling the distribution of electric field at each target. In one embodiment, the lead system includes 2 leads each having 8 electrodes. In another embodiment, the lead system includes individually placed leads, each having 8 or 16 electrodes. Those of ordinary skill in the art will understand that the neurostimulation system 100 may include additional components such as sensing circuitry for direct or indirect patient monitoring and/or feedback control of the therapy, telemetry circuitry, and power. The neurostimulation system 100 may also integrate with other sensors, or such other sensors may independently provide information for use with programming of the neurostimulation system 100, and for data collection and evaluation (e.g., to be considered as part of side effect or treatment efficacy tracking).

The neurostimulation system 100 may be configured to modulate spinal target tissue or other neural tissue. The configuration of electrodes used to deliver electrical pulses to the targeted tissue constitutes an electrode configuration, with the electrodes capable of being selectively programmed to act as anodes (positive), cathodes (negative), or left off (zero). In other words, an electrode configuration represents the polarity being positive, negative, or zero. Other parameters that may be controlled or varied include the amplitude, pulse width, and rate (or frequency) of the electrical pulses. Each electrode configuration, along with the electrical pulse parameters, can be referred to as a “modulation parameter” set. Each set of modulation parameters, including fractionalized current distribution to the electrodes (as percentage cathodic current, percentage anodic current, or off), may be stored and combined into a program that can then be used to modulate multiple regions within the patient.

The neurostimulation system 100 may be configured to deliver different electrical fields to achieve a temporal summation of modulation. The electrical fields can be generated respectively on a pulse-by-pulse basis. For example, a first electrical field can be generated by the electrodes (using a first current fractionalization) during a first electrical pulse of the pulsed waveform, a second different electrical field can be generated by the electrodes (using a second different current fractionalization) during a second electrical pulse of the pulsed waveform, a third different electrical field can be generated by the electrodes (using a third different current fractionalization) during a third electrical pulse of the pulsed waveform, a fourth different electrical field can be generated by the electrodes (using a fourth different current fractionalized) during a fourth electrical pulse of the pulsed waveform, and so forth. These electrical fields can be rotated or cycled through multiple times under a timing scheme, where each field is implemented using a timing channel. The electrical fields may be generated at a continuous pulse rate, or as bursts of pulses. Furthermore, the interpulse interval (i.e., the time between adjacent pulses), pulse amplitude, and pulse duration during the electrical field cycles may be uniform or may vary within the electrical field cycle. In some examples, the modulation field may be shaped to enhance modulation of some neural structures and diminish modulation at other neural structures. The modulation field may be shaped by using multiple independent current control (MICC) or multiple independent voltage control to guide the estimate of current fractionalization among multiple electrodes and estimate a total amplitude that provide a desired strength. For example, the modulation field may be shaped to enhance the modulation of dorsal horn neural tissue and to minimize the modulation of dorsal column tissue.

FIG. 3 illustrates an embodiment of a programming device 302, such as may be implemented in neurostimulation system 100. Programming device 302 represents an embodiment of programming device 102 and includes a storage device 318, a programming control circuit 316, and a user interface device 310. Programming control circuit 316 generates the plurality of stimulation parameters that controls the delivery of the neurostimulation pulses according to the pattern of the neurostimulation pulses. The user interface device 310 represents an embodiment to implement the user interface 110.

In various embodiments, the user interface device 310 includes an input/output device 320 that is capable to receive user interaction and commands to load, modify, and implement neurostimulation programs and schedule delivery of the neurostimulation programs. In various embodiments, the input/output device 320 allows the user to create, establish, access, and implement respective parameter values of a neurostimulation program through graphical selection (e.g., in a graphical user interface output with the input/output device 320), or other graphical input/output relating to therapy and sensing objectives, efficacy of applied treatment, user feedback, and the like. In various examples, the user interface device 310 can receive user input to initiate or control the implementation of the programs or program changes which are recommended, modified, selected, or loaded through use of adaptive neurostimulation (e.g., in response to sensing values), or in connection with other analysis from an open or closed loop programming algorithm or model.

In various embodiments, the input/output device 320 allows a user (e.g., a patient user or, a medical user) to apply, change, modify, or discontinue certain building blocks of a program and a frequency at which a selected program is delivered. In various embodiments, the input/output device 320 can allow the user to save, retrieve, and modify programs (and program settings), such as from programs that are loaded from a clinical encounter or pre-programmed (e.g., as templates). In various embodiments, the input/output device 320 and accompanying software on the user interface device 310 allows newly created building blocks, program components, programs, and program modifications to be saved, stored, or otherwise persisted in storage device 318. Thus, it will be understood that the user interface device 310 may allow many forms of device operation and control, even if automated (e.g., adaptive or closed loop) programming is occurring.

The user interface device 310 may provide an interactive mechanism, controllable with the input/output device 320, for the entry, selection, verification, or indication of therapy and sensing programming objectives. In some examples, this includes a user interface control to enter or change the weights or values that select or indicate an amount of balance between a priority of therapy objectives and a priority of sensing objectives. Examples of such a user interface control are provided in FIG. 7, discussed below.

In one embodiment, the input/output device 320 includes a touchscreen. In various embodiments, the input/output device 320 includes a presentation device, such as interactive or non-interactive screens, and a user input device that allows the user to interact with a user interface to implement or change programming. Thus, the input/output device 320 may include one or more of a touchscreen, keyboard, keypad, touchpad, trackball, joystick, and mouse. The logic of the user interface 110, the stimulation control circuit 214, and the programming control circuit 316, including their various embodiments discussed in this document, may be implemented using an application-specific circuit constructed to perform one or more particular functions or a general-purpose circuit programmed to perform such function(s). Such a general-purpose circuit includes, but is not limited to, a microprocessor or a portion thereof, a microcontroller or portions thereof, and a programmable logic circuit or a portion thereof.

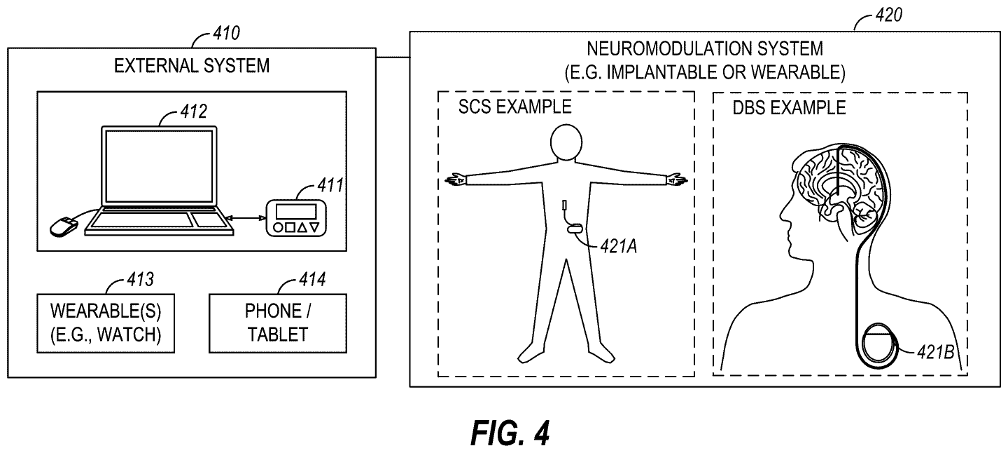

FIG. 4 illustrates an implantable neurostimulation system 420 and portions of an environment in which system 420 may be used, such as may be implemented as the stimulation device 104, 204 illustrated in FIGS. 1 and 2. FIG. 4 specifically illustrates, by way of example and not limitation, the neurostimulation system 100 of FIG. 1 implemented in a spinal cord stimulation (SCS) system or a deep brain stimulation (DBS) system. The illustrated neuromodulation system 420 connects with an external system 410 that may include at least one programming device. The illustrated external system 410 may include a programmer 412 (e.g., clinician programmer) configured for use by a clinician to communicate with and program the neurostimulator, and a remote control device 411 configured for use by the patient to communicate with and program the neurostimulator. For example, the remote control device 411 may allow the patient to turn a therapy on and off and/or may allow the patient to adjust patient-programmable parameter(s) of the modulation parameters (e.g., by switching programs).

FIG. 4 further illustrates the neuromodulation system 420 as an ambulatory medical device, such as implemented by stimulation device 421A or stimulation device 421B. Examples of ambulatory devices include wearable or implantable neuromodulators. The external system 410 may include a network of computers, including computer(s) remotely located from the ambulatory medical device that are capable of communicating via one or more communication networks with the programmer 412 and/or the remote control device 411. The remotely located computer(s) and the ambulatory medical device may be configured to communicate with each other via another external device such as the programmer 412 or the remote control device 411.

The external system 410 may also include one or more wearables 413 and a portable device 414 such as a smartphone or tablet. In some examples, the wearables 413 and the portable device 414 may allow a user to obtain and provide input data, such as external sensor data values (e.g., from a physiologic sensor of a wearable) or feedback/status information (e.g., on a phone/tablet screen) for data collection. In some examples, the remote control device 411 and/or the programmer 412 also may display recommendations or program settings (e.g., derived from a sensor-driven adaptive treatment, or produced from other variations of a fully or partially closed-loop programming algorithm or model). The remote control device 411 and/or the programmer 412 may be used to provide other aspects of input and output, including inputs from the usage data of various neurostimulation programs, events associated with such programs, and the like.

As used herein, the terms “neurostimulator,” “stimulator,” “neurostimulation,” and “stimulation” generally refer to the delivery of electrical energy that affects the neuronal activity of neural tissue, which may be excitatory or inhibitory; for example by initiating an action potential, inhibiting or blocking the propagation of action potentials, affecting changes in neurotransmitter/neuromodulator release or uptake, and inducing changes in neuro-plasticity or neurogenesis of tissue. It will be understood that other clinical effects and physiological mechanisms may also be provided through use of such stimulation techniques.

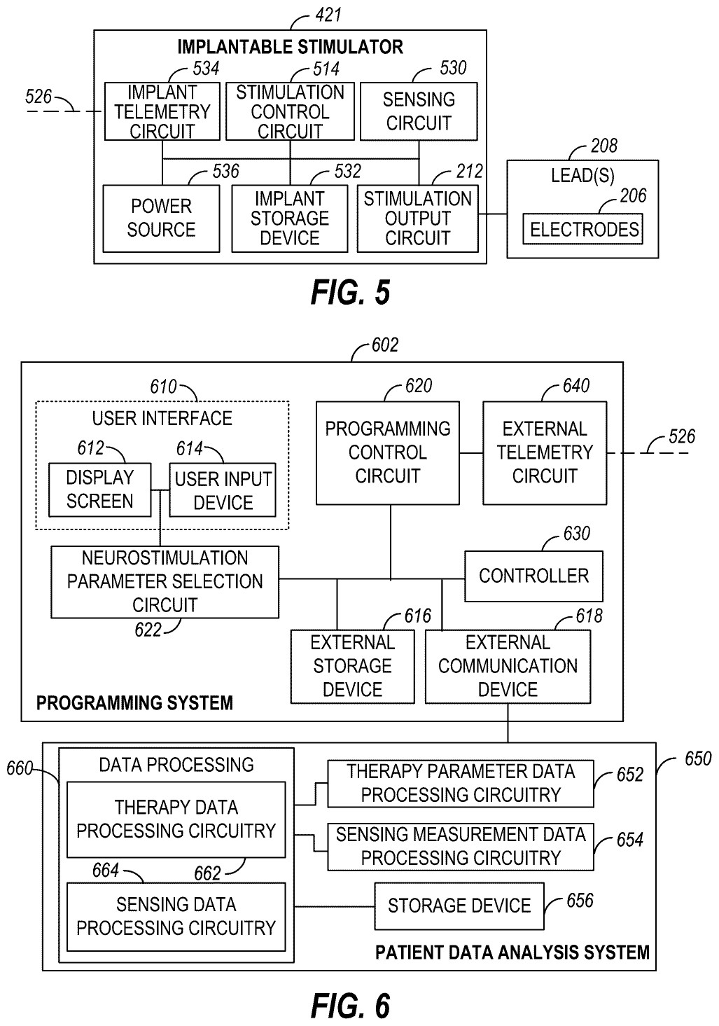

FIG. 5 illustrates an embodiment of the implantable stimulator 421 and the one or more leads 208 of an implantable neurostimulation system, such as the implantable system 420. The implantable stimulator 421 may include a sensing circuit 530 used for sensing functions, stimulation output circuit 212, a stimulation control circuit 514, an implant storage device 532, an implant telemetry circuit 534, and a power source 536. The sensing circuit 530, when included, senses one or more physiological signals for purposes of patient monitoring and/or feedback control of the neurostimulation (e.g., via automated, semi-automated, or manual control). Examples of sensing the physiological signals includes neural and other signals that are indicative of a condition of the patient that is treated by the neurostimulation and/or a response of the patient to the delivery of the neurostimulation therapy. Such physiological signals include neural activity that is used to determine ECAP and other measurements.

The stimulation output circuit 212 is electrically connected to electrodes 206 through the one or more leads 208, and delivers each of the neurostimulation pulses through a set of electrodes selected from the electrodes 206. The stimulation output circuit 212 can implement, for example, the generating and delivery of a customized neurostimulation waveform (e.g., implemented from a parameter of a selected or specified program) to an anatomical target of a patient.

The stimulation control circuit 514 represents an embodiment of the stimulation control circuit 214 and controls the delivery of the neurostimulation pulses using the plurality of stimulation parameters specifying the pattern of the neurostimulation pulses. In one embodiment, the stimulation control circuit 514 directly or indirectly controls the delivery of the neurostimulation pulses using the one or more sensed physiological signals and processed input from patient feedback interfaces. The implant telemetry circuit 534 provides the implantable stimulator 421 with wireless communication with another device such as a device of the external system 410, including receiving values of the plurality of stimulation parameters from the external system 410. The implant storage device 532 stores values of the plurality of stimulation parameters, including parameters from one or more programs which are activated, de-activated, or modified using the approaches discussed herein.

The power source 536 provides the implantable stimulator 421 with energy for its operation. In one embodiment, the power source 536 includes a battery. In one embodiment, the power source 536 includes a rechargeable battery and a battery charging circuit for charging the rechargeable battery. The implant telemetry circuit 534 may also function as a power receiver that receives power transmitted from external system 410 through an inductive couple.

In various embodiments, the sensing circuit 530, the stimulation output circuit 212, the stimulation control circuit 514, the implant telemetry circuit 534, the implant storage device 532, and the power source 536 are encapsulated in a hermetically sealed implantable housing. In various embodiments, the lead(s) 208 are implanted such that the electrodes 206 are placed on and/or around one or more targets to which the neurostimulation pulses are to be delivered, while the implantable stimulator 421 is subcutaneously implanted and connected to the lead(s) 208 at the time of implantation.

FIG. 6 illustrates an embodiment of a programming system 602 used as part of an implantable neurostimulation system, such as the external system 420, with the programming system 602 configured to send and receive device data (e.g., commands, parameters, program selections, information). FIG. 6 also illustrates an embodiment of a patient data analysis system 650, communicatively coupled to the programming system 602. The patient data analysis system 650 is used to evaluate sensing and therapy states occurring in the patient, to identify changes and adaptations to the therapy that can improve sensing capabilities, and to determine specific parameters, parameter changes, or programming values in connection with planned or ongoing neurostimulation treatment by the implantable neurostimulation system.

The programming system 602 represents an embodiment of the programming device 302, and includes an external telemetry circuit 640, an external storage device 616, a programming control circuit 620, a user interface device 610, a controller 630, and an external communication device 618, to effect programming of a connected neurostimulation device. The operation of the neurostimulation parameter selection circuit 622 enables selection, modification, and implementation of a particular set of parameters or settings for neurostimulation programming (e.g., via selection of a program, specification by an adaptive programming process, selection by a patient or clinician for testing or experimentation, or the like).

The external telemetry circuit 640 provides the programming system 602 with wireless communication to and from another controllable device such as the implantable stimulator 421 via a telemetry link 526, including transmitting one or a plurality of stimulation parameters (including selected, identified, or modified stimulation parameters of a selected program) to the implantable stimulator 421. In one embodiment, the external telemetry circuit 640 also transmits power to the implantable stimulator 421 through inductive coupling.

The external communication device 618 may provide a mechanism to conduct communications with a programming information source, such as a data service, program modeling system, to receive program information, settings and values, models or algorithmic data rules, functionality controls, or the like, via an external communication link (not shown). In a specific example, the external communication device 618 communicates with the patient data analysis system 650 to identify parameters or settings that are selected, modified, or implemented for the neurostimulation programming. The external communication device 618 may communicate using any number of wired or wireless communication mechanisms described in this document, including but not limited to IEEE 802.11 (Wi-Fi), Bluetooth, Infrared, and like standardized and proprietary wireless communications implementations. Although the external telemetry circuit 640 and the external communication device 618 are depicted as separate components within the programming system 602, the functionality of both of these components may be integrated into a single communication chipset, circuitry, or device.

The external storage device 616 stores a plurality of existing neurostimulation waveforms, including definable waveforms for use as a portion of the pattern of the neurostimulation pulses, settings and setting values, other portions of a program, and related treatment information. In various embodiments, each waveform of the plurality of individually definable waveforms includes one or more pulses of the neurostimulation pulses, and may include one or more other waveforms of the plurality of individually definable waveforms. Examples of such waveforms include pulses, pulse blocks, pulse trains, and train groupings, and programs. The existing waveforms stored in the external storage device 616 can be definable at least in part by one or more parameters including, but not limited to the following: amplitude, pulse width, frequency, duration(s), electrode configurations, total charge injected per unit time, cycling (e.g., on/off time), waveform shapes, spatial locations of waveform shapes, pulse shapes, number of phases, phase order, interphase time, charge balance, and ramping.

The external storage device 616 may also store a plurality of individually definable fields that may be implemented as part of a program. Each waveform of the plurality of individually definable waveforms is associated with one or more fields of the plurality of individually definable fields. Each field of the plurality of individually definable fields is defined by one or more electrodes of the plurality of electrodes through which a pulse of the neurostimulation pulses is delivered and a current distribution of the pulse over the one or more electrodes. A variety of settings in a program may be correlated to the control of these waveforms and definable fields.

The programming control circuit 620 represents an embodiment of a programming control circuit 316 and may translate or generate the specific stimulation parameters or changes which are to be transmitted to the implantable stimulator 421, based on the results of the neurostimulation parameter selection circuit 622. The pattern may be defined using one or more waveforms selected from the plurality of individually definable waveforms (e.g., defined by a program) stored in an external storage device 616. In various embodiments, the programming control circuit 620 checks values of the plurality of stimulation parameters against safety rules to limit these values within constraints of the safety rules. In one embodiment, the safety rules are heuristic rules.

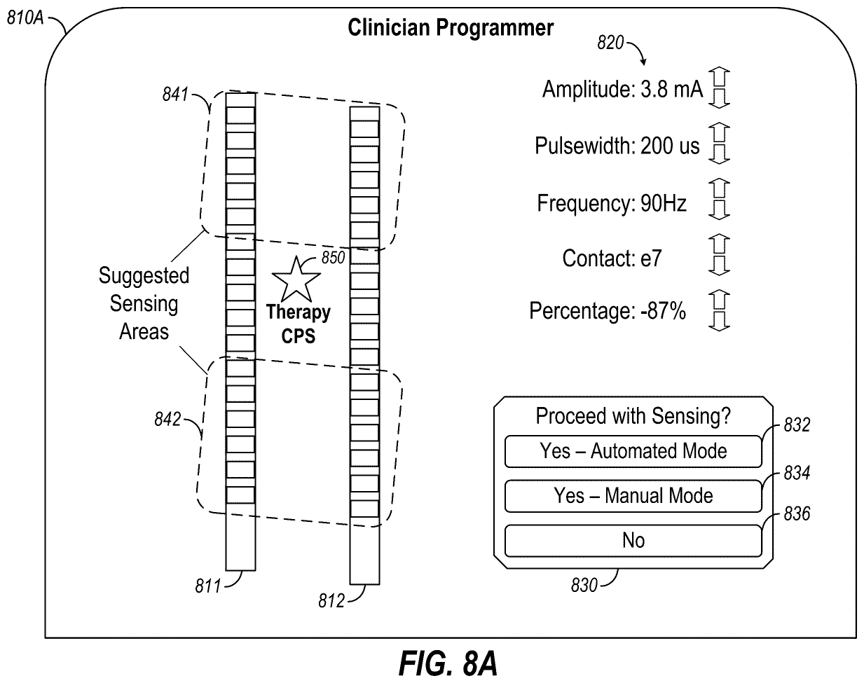

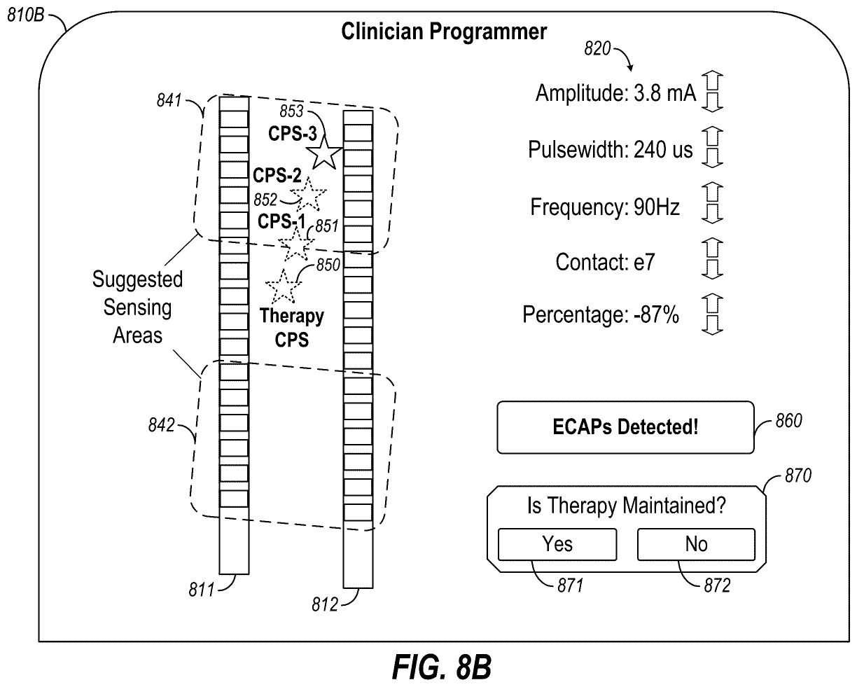

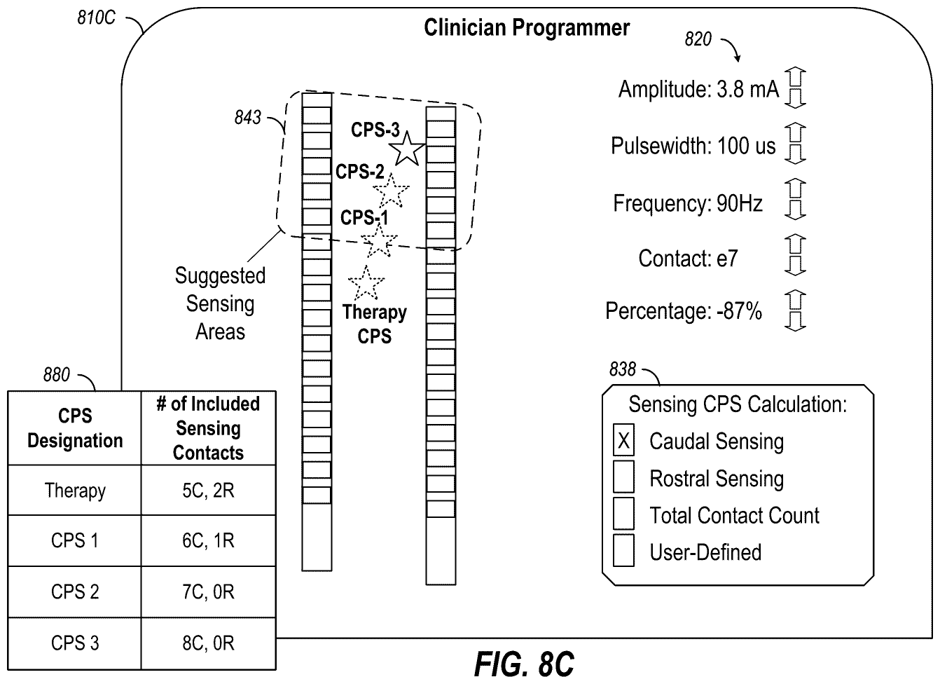

The user interface device 610 represents an embodiment of the user interface device 310 and allows the user (including a patient or clinician) to provide input relevant to sensing and therapy objectives, including to customize adaptive sensing-driven programming that modifies programs or changes operational use of the programs. The user interface device 610 includes a display screen 612, a user input device 614, and may implement or couple to the neurostimulation parameter selection circuit 622, or data provided from the patient data analysis system 650. The display screen 612 may include any type of interactive or non-interactive screens, and the user input device 614 may include any type of user input devices that supports the various functions discussed in this document, such as a touchscreen, keyboard, keypad, touchpad, trackball, joystick, and mouse. The user interface device 610 may also allow the user to perform other functions where user interface input is suitable (e.g., to select, modify, enable, disable, activate, schedule, or otherwise define a program, sets of programs, provide feedback or input values, or perform other monitoring and programming tasks). Although not shown, the user interface device 610 may also generate a visualization of such characteristics of device implementation or programming, and receive and implement commands to implement or revert the program and the neurostimulator operational values (including a status of implementation for such operational values). These commands and visualization may be performed in a review and guidance mode, testing or experimentation mode, status mode, or in a real-time programming mode. Consistent with the examples provided herein, the user interface device 610 may provide user interface screens to represent or change the location of programming effects, including sensing capabilities or the delivery of therapy as discussed below (e.g., with reference to FIGS. 8A to 13).

The controller 630 can be a microprocessor that communicates with the external telemetry circuit 640, the external communication device 618, the external storage device 616, the programming control circuit 620, the neurostimulation parameter selection circuit 622, and the user interface device 610, via a bidirectional data bus. The controller 630 can be implemented by other types of logic circuitry (e.g., discrete components or programmable logic arrays) using a state machine type of design. As used in this disclosure, the term “circuitry” should be taken to refer to discrete logic circuitry, firmware, or to the programming of a microprocessor.

The patient data analysis system 650 is configured to operate data processing circuitry 660, which may include therapy data processing circuitry 662 that identifies properties (e.g., type, timing, location) of one or more neurostimulation treatment parameters relevant to causing therapy or therapy effects, and sensing data processing circuitry 664 that identifies properties (e.g., type, timing, location) of the one or more neurostimulation treatment parameters relevant to sensing capabilities. The therapy data processing circuit 662 may model the use of particular neurostimulation programming parameters (and parameter values) using therapy parameter data processing circuitry 652 (e.g., by analyzing patient responses to therapy). The sensing effects data processing circuitry 664 may model the use of the particular neurostimulation programming parameters (and parameter values) using sensing measurement data processing circuitry 654 (e.g., by analyzing neural activity measurements).

Some data values and neurostimulation programming may be automatically determined, recommended, or adjusted based on adaptive (e.g., sensing-data driven) programming changes, including with closed-loop or patient-responsive (i.e., partially-closed-loop) programming approaches. For instance, the patient data analysis system 650 may evaluate sensing data (via the sensing measurement data processing circuitry 654, and the sensing effects data processing circuitry 664) obtained from the neurostimulation treatment, as discussed in more detail below. The patient data analysis system 650 may also evaluate sensor data from one or more patient sensors (e.g., wearables, sleep trackers, motion tracker, implantable devices, etc.) among one or more internal or external devices. The sensor data may provide medical data to determine a customized and current state of the patient condition or neurostimulation treatment results, for adaptive or dynamic changes.

In various examples, the stimulator 421 includes sensors which contribute to the sensor data to be evaluated by the patient data analysis system 650. In an example, the patient sensors are physiological or biopsychosocial sensors that collect data relevant to physical, biopsychosocial (e.g., stress and/or mood biomarkers), or physiological factors relevant to a state of the patient. Examples of such sensors might include a sleep sensor to sense the patient's sleep state (e.g., for detecting lack of sleep), a respiration sensor to measure patient breathing rate or capacity, a movement sensor to identify an amount or type of movement, a heart rate sensor to sense the patient's heart rate, a blood pressure sensor to sense the patient's blood pressure, an electrodermal activity (EDA) sensor to sense the patient's EDA (e.g., galvanic skin response), a facial recognition sensor to sense the patient's facial expression, a voice sensor (e.g., microphone) to sense the patient's voice, and/or an electrochemical sensor to sense stress biomarkers from the patient's body fluids (e.g., enzymes and/or ions, such as lactate or cortisol from saliva or sweat). Other types or form factors of sensor devices may also be utilized.

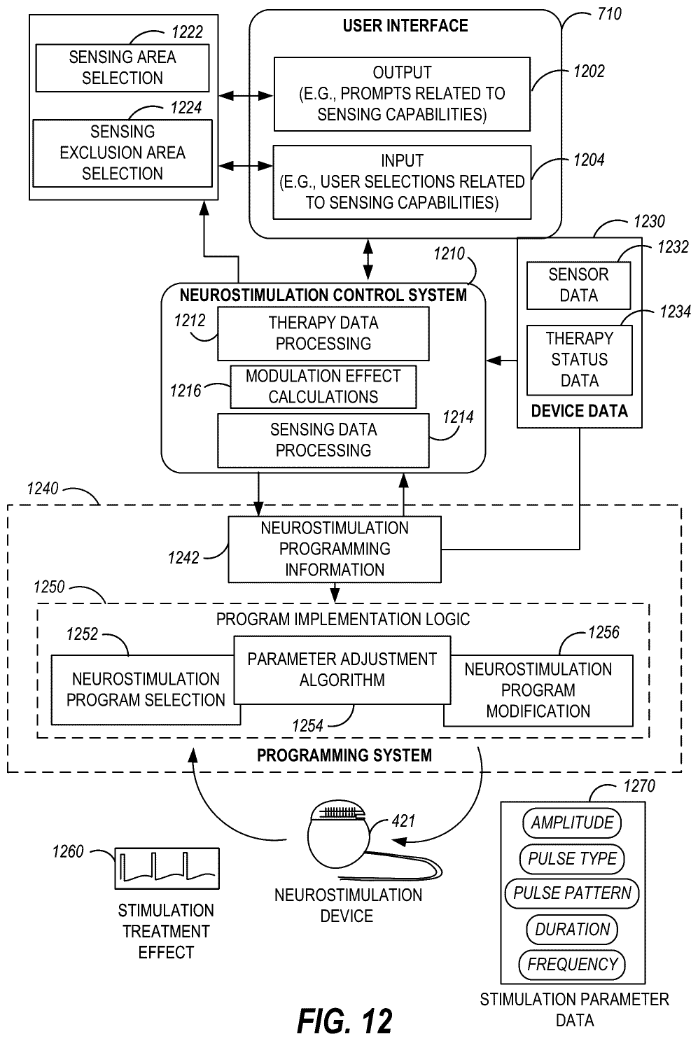

The patient data analysis system 650 also is depicted as including a storage device 656 to store or persist data related to the sensing or therapy functions, and for associated medical data, device data, patient or clinician input and output, and related settings, logic, or algorithms. Other hardware features of the patient data analysis system 650 are not depicted for simplicity, but are suggested from functional capabilities and operations in the following figures. Additional detail regarding adaptive or closed-loop programming and control of the stimulator 421, based on user input of therapy and sensing objectives and changed programming parameters, are provided in the example of FIG. 12, below. However, it will be understood that other functionality for programming therapy modeling and sensing adaptation, and related program modeling, selection, recommendation, and implementation, may be provided via programming devices, data services, or information services that are not depicted.

FIG. 7 illustrates, by way of example, a user interface 710 on an example user computing device 700, which may provide an implementation of the user interface device 610 discussed above. The user interface 710 may enable user control and initiation of a programming session, including the user control of therapy and sensing objectives for neurostimulation treatments. Although not depicted, the user interface 710 may also present and receive information for other aspects of programming and control of neurostimulation (including features of the programmer 412, programming system 602, and/or the patient data analysis system 650). In an example, the computing device 700 is a computing device (e.g., personal computer, tablet, smartphone) or other user-operated device that receives and provides interaction from a patient, caregiver, clinician, or other person via the graphical user interface 710.

The user interface 710, as depicted, includes two graphical user interface input features 720, 730 to receive input for the selection of therapy and sensing objectives. In many examples, only one of these input features may be presented. Other variations, such as different types of controls or screen layouts, may be provided. The user computing device 700 may include a touchscreen or similar input feature to toggle or change the features 720, 730.

The input feature 720, as depicted, includes multiple selection boxes to receive a binary selection of one or more programming objectives from a user. A therapy selection box 722 may be selected or de-selected to instruct the system to prioritize a therapy objective. A sensing selection box 724 may be selected or de-selected to instruct the system to prioritize a sensing objective. The selection of both boxes may indicate an equal or balanced objectives. The user may confirm the selection via the button 726.

The input feature 730 is depicted as including a slider 732 to select one of multiple selection options, to collect input for programming objectives from a user. For example, a selected slider position 734 may correspond to a therapy focus exclusively, a sensing focus exclusively, or some balance (emphasizing therapy over sensing, emphasizing sensing over therapy, etc.). The user may confirm a selection of the inputs via the button 736. In connection with the central point of stimulation (CPS) identification process depicted in FIGS. 8A and 8B, either of the input features 720, 730 enable a change of a CPS, to determine a value for a CPS emphasizing a location with a therapy focus and a CPS emphasizing a location with a sensing focus.

FIG. 8A illustrates, by way of example, a user interface 810A to provide information relevant to programming areas and locations. This user interface 810A is specifically shown as part of a clinician programmer screen, but may be incorporated into other aspects of a patient programmer or other graphical user interface platforms.

The user interface 810A includes a depiction of an arrangement of two leads (a first lead 811 and a second lead 812) and electrode contacts on the lead. The user interface 810A also includes a listing of parameters 820, such as amplitude, pulse width, and frequency, along with an identification of a contact and percentage for the parameter values. In some examples, the listing of parameters 820 may be accompanied with user interface controls such as buttons or inputs (e.g., arrow buttons) to change (e.g., increase or decrease) or to enter values for the parameters. For instance, “Percentage” represents the fraction (or “fractionalization”) of the total amplitude that is configured to be delivered through a given contact, and this value can be adjusted upwards or downwards using the respective arrow buttons (or a similar interface control). In other examples, the listing of parameters 820 is provided in a read-only mode, as the user interface displays a current or recommended parameter value.

The user interface 810A may initiate and display a determination of a CPS for a particular set of therapy parameters, and to confirm a patient report of therapeutic effect from the use of the therapy parameters (e.g., using user interface functions provided by sensing-agnostic programming). In the user interface 810A, a location of the therapy CPS 850 is depicted, to correspond to a calculated or estimated CPS based on the parameters. As will be understood, an electrode configuration and the use of particular parameters with the electrode configuration are used to specify a CPS to be provided at a specific location in the patient's tissue.

Based on the location of the CPS and whether sensing capabilities can be detected, the clinician programmer may initiate a process of reconciling a therapy-only CPS with a therapy-and-sensing CPS or with a sensing-only CPS. This may include the clinician programmer initiating an automated or manual process to guide a user in a procedure to find a new CPS that balances these objectives. As used herein, a balanced CPS refers to a CPS location that provides therapy outcomes (to deliver neurostimulation effects from the electrical contacts, such as pain treatment according to treatment or clinical objectives) but which also supports sensing capabilities (to capture data from the electrical contacts, which represents neural activity elicited or caused by the neurostimulation). This procedure to find a new CPS may attempt to equally balance sensing and therapy, but in other examples may also favor sensing over therapy or therapy or sensing (e.g., as depicted in the user inputs of FIG. 7).

The settings that are used to guide a search for a new CPS may be based on one or more suggested or recommended sensing areas, such as the suggested sensing areas 841, 842 depicted in FIG. 8A. A series of user input buttons are provided to control the automated or manual process for adjusting parameters and changing the CPS to a new location in the suggested sensing areas. A first button 832 can be activated to proceed with the selected settings and enable selection of an automated sensing mode, to automatically identify parameter changes that improve the locations of available sensing. A second button 834 can be activated to proceed with the selected settings and enable selection of a manual mode, to identify locations of available sensing from user control (e.g., as the user changes parameter values). A third button 836 can be activated to reject the settings or to avoid use of a sensing mode. Other user interface features to preview the CPS actions or sensing area effects may also be provided.

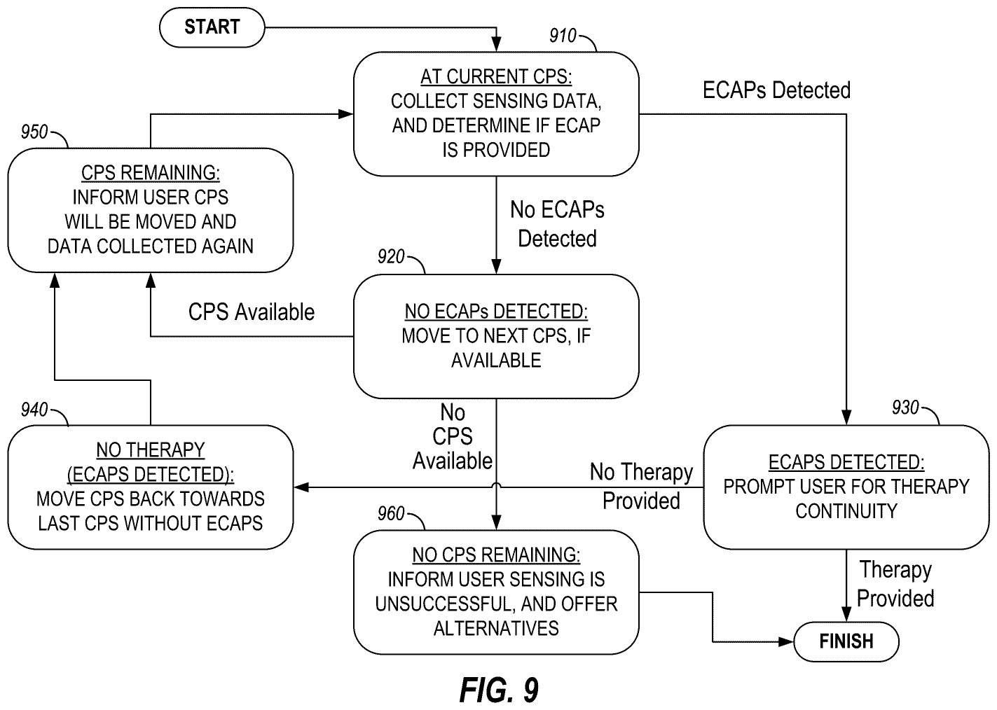

As an example of a manual programming process (e.g., selected via button 834), a data sample will be collected (using appropriately selected sensing channels) when reaching a stationary CPS. An outcome is then displayed to the user regarding the amount or type of sensing capabilities at detected at the CPS (e.g., whether one or more ECAPs have been detected at the CPS location). In an automated programming process (e.g., selected via button 832), the process may automatically evaluate sensing capabilities at a number of CPS locations, and automatically move to new CPS locations as detailed by the flowchart in FIG. 9, discussed below. Either sensing reconciliation process can be guided by a user (e.g., clinician, caregiver, patient) who ensures that therapy is maintained while cycling through sensing options and new CPS locations.

FIG. 8B illustrates, by way of example, a user interface 810B of the clinician programmer which is adapted to provide control of an automated or manual programming process. This user interface 810B includes the features discussed with reference to FIG. 8A, but also demonstrates the outcomes of changing the CPS from a first location (Therapy CPS 850) to a second location (CPS-1 851), to a third location (CPS-2 852), and finally to a fourth location (CPS-3 853). In an automated scenario, the movement from the first to second to third to fourth location may occur automatically, to iteratively change the parameters and continue the movement of the CPS until an evoked compound action potential (ECAP) is observable at the final location (CPS-3 853). In a manual scenario, a sequence of user inputs and interactions may be received that move the CPS to each location (e.g., as a result of a user changing the pulse width value, which is then activated and tested).

The user interface 810B also includes an information display 860 to output a message when ECAPs are detected, and a selectable option 870 for the user to input whether therapy is maintained. The selectable option 870 is shown as including a binary control (e.g., a “Yes” button 871 and a “No” button 872). In many use cases, the user is responsible for checking with the patient that suitable therapy results (e.g., pain control) have been maintained. Thus, in the example of FIG. 8B, a clinician may activate one of the selectable options 870 after observing and verifying with the patient.

FIG. 8C illustrates, by way of example, a user interface 810C of the clinician programmer that is adapted to reconcile locations which identify a CPS based on particular designations of sensing contacts. Inclusion/exclusion count at a given set of parameters may be used to determine optimum sensing CPS designations, e.g. by geometry and stimulation phase width. For example, this may be performed by counting a number of available rostral (R), caudal (C), and/or total contacts, and identifying a new CPS with maximum available counts. This user interface also includes a selection option 838 used to control a sensing CPS calculation based on one or more designations or priorities such as based on: caudal sensing; rostral sensing; total contact count; or other user-defined settings.

In the example of FIG. 8C, a data set 880 includes a listing of a number of sensing contacts for multiple CPS locations. For instance, starting at a therapy CPS location, this data set 880 indicates a count of contacts, e.g., 5 caudal and 2 rostral contacts, which may be used for sensing. This data set 880 may vary or be updated by waveform per process. The data set 880 indicates that at CPS 1 location, 6 caudal and 2 rostral contacts may be used for sensing; at a CPS-2 location, 7 caudal and 1 rostral contacts may be used for sensing; at a CPS-3 location, 8 caudal and 0 rostral contacts may be used for sensing. Because caudal sensing is selected in selection option 838, the CPS location will be moved into the suggested sensing area 843 to a location that maximizes the number of caudal sensing contacts. This results in CPS-3 being selected, since it includes the highest number of caudal contacts used for sensing. In a further example, the count of contacts may be stored or computed in a table, and multiple tables may be pre-loaded based on the type of waveform or waveform changes (e.g., to identify optimized CPSes across different waveform conditions).

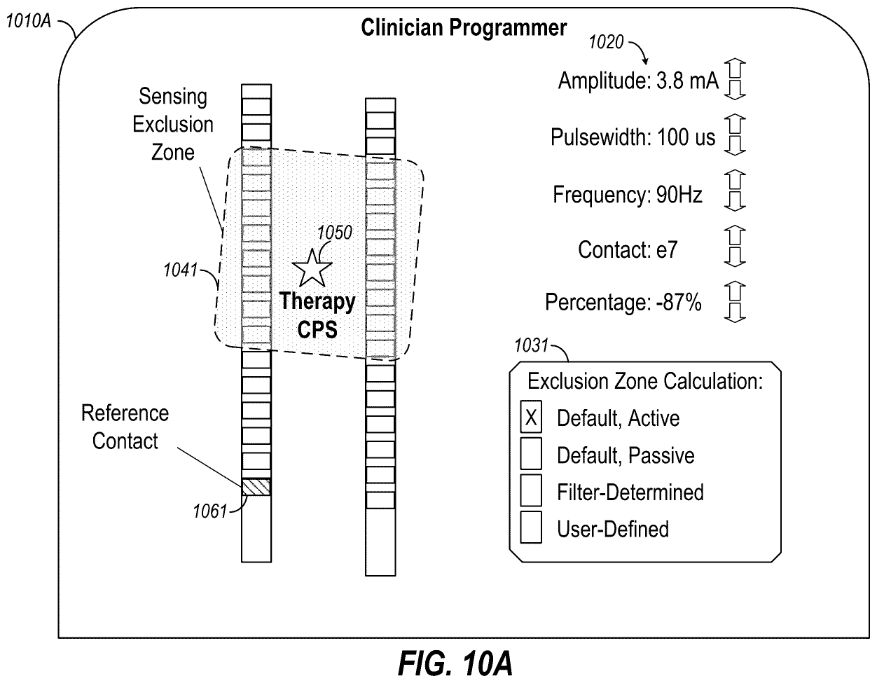

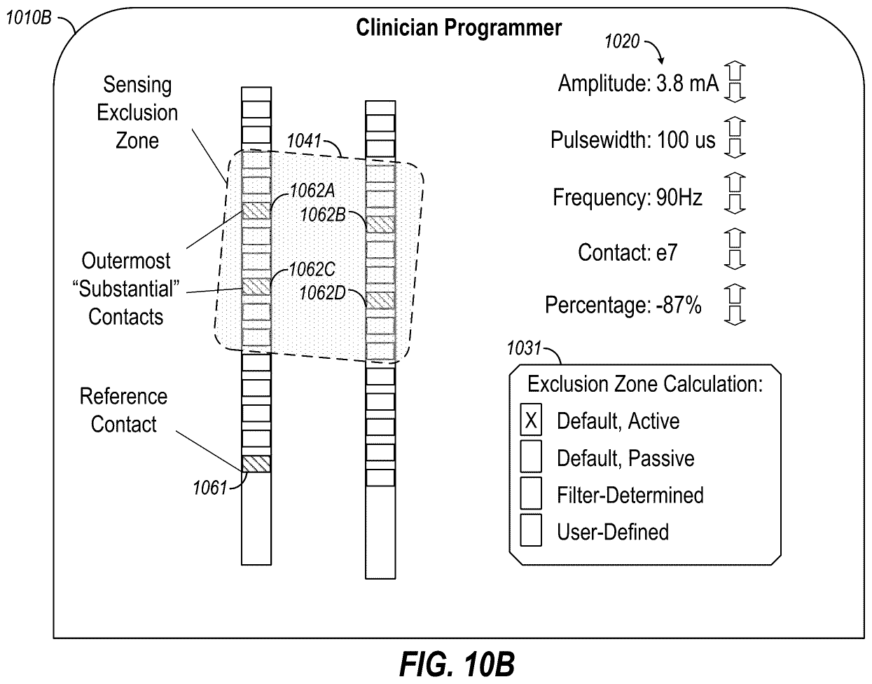

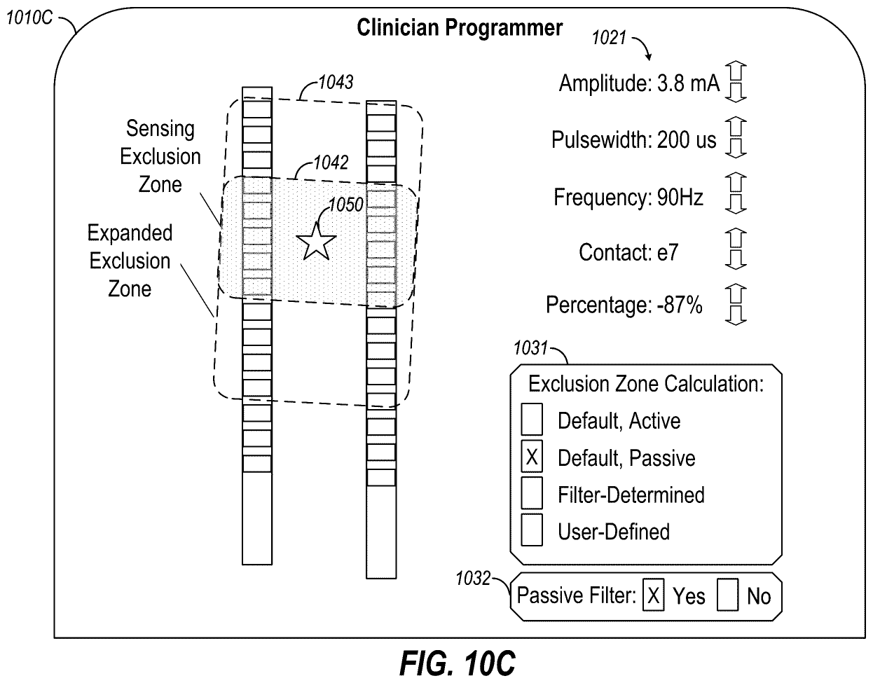

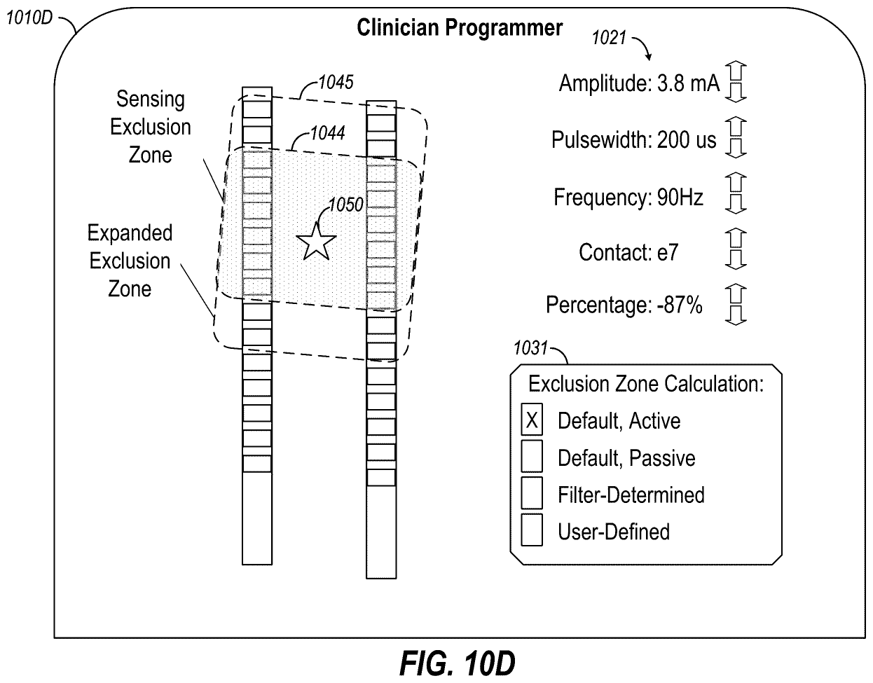

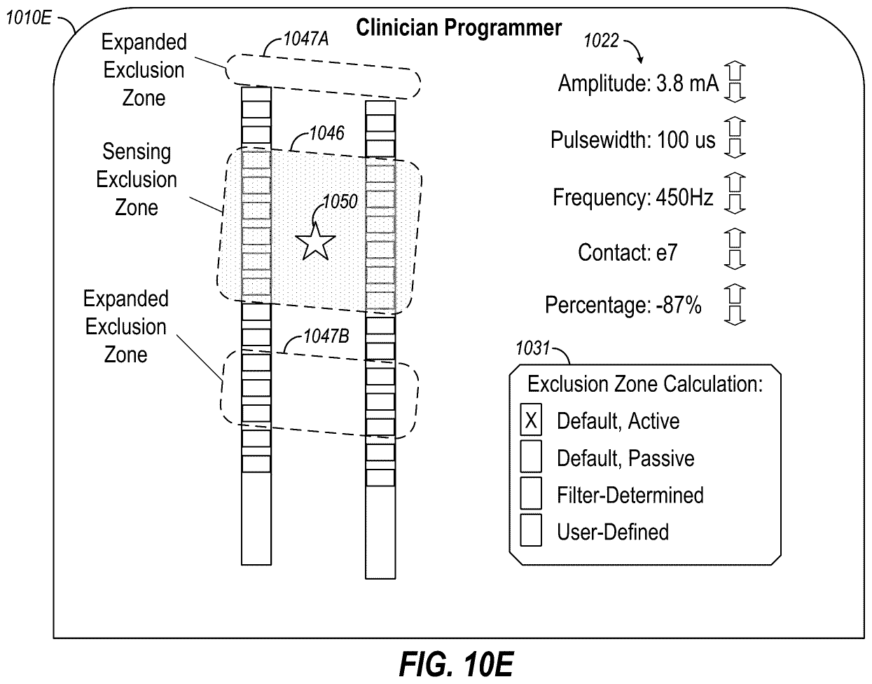

The approach used in FIG. 8C may also be modified based on exclusionary areas that identify areas in which sensing is not available. Additional discussion of sensing exclusion areas and user interfaces that provide capabilities based on sensing exclusion areas or zones are provided below with reference to FIGS. 10A to 10E.

FIG. 9 depicts a flowchart of an example process for automated sensing reconciliation. As noted, this process may be started with feedback a user to begin the automated selection, including the user selection of starting parameters. Thus, this process may be invoked based on features provided in the user interfaces 810A, 810B, 810C as discussed above.

Operation 910 includes collecting sensing data at a current CPS, and determining if sensing capabilities are provided the current CPS. The sensing capabilities may be evaluated based on an assessment of an ECAP or another potential measurement (e.g., to determine if a physiological measurement of the stimulation treatment can be sensed at the current CPS). If one or more ECAPs are detected, then the process proceeds to operation 930, but if no ECAPs are detected, then the process proceeds to operation 920.

Operation 920, which occurs when no ECAPs are detected, includes moving to a next CPS, if another CPS is available for selection. If another CPS is available, the process proceeds to operation 950. If another CPS is not available, the process proceeds to operation 960.

Operation 930, which occurs when ECAPs are detected, includes prompting a user to determine if therapy continuity has been provided. This may include presenting a selectable option in a clinician programmer or other user interface, to ask if the therapy results are being provided at the current CPS. If therapy is provided, then the process ends with identifying a CPS with therapy and sensing capabilities. If therapy is not provided, then the process proceeds to operation 940. In some examples, a configuration that generates an ECAP may be saved for future use (or interleaved use), regardless of whether therapy is or is not provided.

Operation 940, which occurs when ECAPs are detected but therapy is not provided, includes moving a CPS back to a previous setting (e.g., towards the last CPS without ECAPs). The process then proceeds to operation 950.

Operation 950, which occurs when ECAPs are not detected, includes informing the user that the CPS will be moved to a new location (a new CPS that becomes the current CPS). This is followed by repeating operation 910.

Operation 960, which occurs when no CPS locations are remaining, includes informing the user that sensing is unsuccessful. In further examples, this may be followed by the recommendation or use of alternative programming strategies, such as interleaved therapy and pulse configurations that enable combined sensing and therapy operations.

FIG. 10A illustrates, by way of example, a user interface 1010A of the clinician programmer which is adapted to incorporate features of a sensing exclusion zone. Based on information about the waveform used for neurostimulation, a sensing exclusion zone can be highlighted or shaped around a given CPS. This sensing exclusion zone can represent an area or areas in which sensing cannot occur (or is unlikely to occur), relative to a CPS or another therapy location such as an initial therapy CPS 1050.

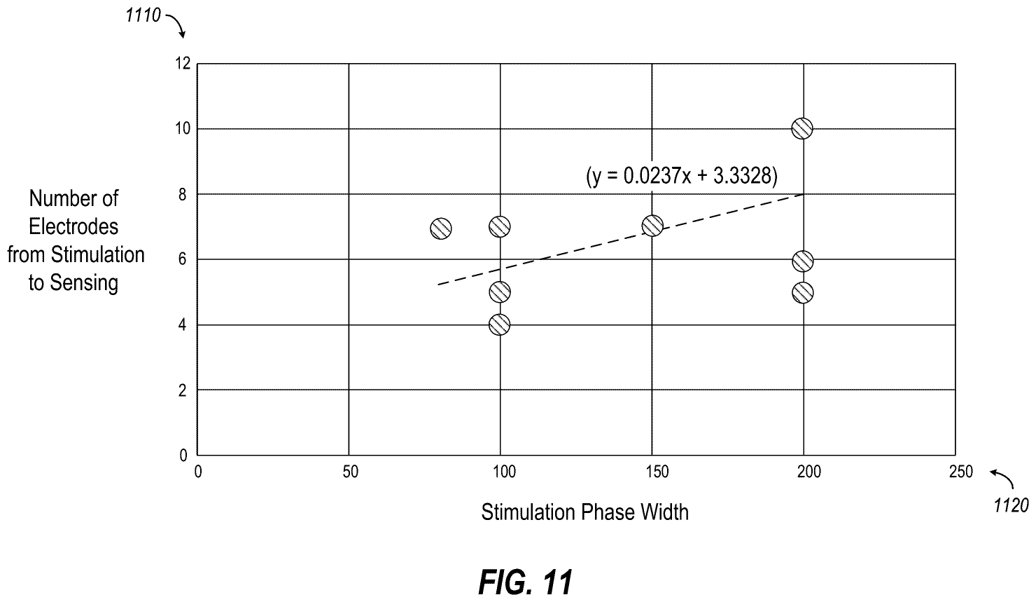

For instance, a sensing exclusion area 1041 (shown with a dashed box) represents an estimate of where sensing would not be possible, given the stimulation waveform resulting from the parameters 1020. This sensing exclusion area 1041 may be derived from a relationship between stimulation phase width and number of electrodes (discussed in more detail in FIG. 11, below), with the area 1041 rotated slightly due to lead orientation. The shape of the sensing exclusion area 1041 may be derived more broadly from assumed or measured conduction velocities of sensing elements (e.g. dorsal column fibers), among other input data. For instance, measurements of ECAPs can produce conduction velocity estimates that can be used to determine the parameters of the sensing exclusion area on a patient-specific or programming session-specific basis. In some examples, the shape of the sensing exclusion area 1041 may be determined by waveform shape (e.g. active vs. passive), a linear assumption (e.g., a “default” value) versus an estimated value based on different sensing filters, or based on user-defined calculations and setting. A selection of one or more of these calculations may be provided based on a selection of one or more calculation options 1031.

Similar to the functionality discussed above, the user interface 1010A may include a listing of parameters 1020 and functionality to change the parameters. The user interface 1010A may also provide other capabilities to identify and interact with the sensing exclusion area 1041 or therapy CPS 1050, or to display other information relating to sensing capabilities or treatment locations. This may include the illustration (e.g., highlighting) or designation of reference contacts such as reference contact 1061, disconnected contacts, and/or contacts where sensing is otherwise not possible outside of exclusion zone.

FIG. 10B illustrates, by way of example, another user interface 1010B of the clinician programmer. As in FIG. 10A, this user interface shows a listing of parameters 1020, calculation options 1031, and a representation of a sensing exclusion area 1041. However, in this example, the sensing exclusion area 1041 may be calculated from an “outermost” substantial (magnitude of fractionalization>25%) contact from CPS rather than/in addition to therapy CPS. In the user interface 1010B, such substantial contacts may include those depicted in 1062A, 1062B, 1062C, 1062D. Other contacts represent those on which sensing may be feasible (assuming a far reference). The impact of artifacts may be scaled by the fractionalization. Accordingly, the calculation of a contact-based exclusion zone may be same, or different, from a CPS-based calculation.

FIG. 10C illustrates, by way of example, another user interface 1010C of the clinician programmer. As in FIG. 10A, this user interface shows a listing of parameters 1021 (with an increased pulse width from parameters 1020) and calculation options 1031. If the exclusion zone calculation is based on a “Default, Passive” selection, then the exclusion zone may depend on whether or not an additional artifact filter is applied (e.g., an additional artifact filter that subtracts off a long passive recharge period). An option for selecting the passive filter is shown with selection options 1032. If the passive artifact filter is applied, then size of the sensing exclusion area 1042 may be reduced as shown (e.g., half of waveform phase width). If the passive artifact filter is not applied, then the sensing exclusion area may be expanded to “wait out” the most impactful passive recharge components, shown with expanded sensing exclusion area 1043. In an example, the “Yes” option of the passive filter selection options 1032 may be selected by default; or, a user may decide based on a waveform visualization.

FIG. 10D illustrates, by way of example, a user interface 1010D of the clinician programmer. As in FIG. 10C, this user interface shows a listing of parameters 1021 and calculation options 1031. Here, the sensing exclusion area 1044 represents an estimate of where sensing would not be possible, based on an active calculation of the stimulation waveform. Increasing the pulse width to 200 us, however, will expand the size of stimulation artifacts and thereby expand a spatial exclusion zone corresponding to the propagation distance equivalent to time expansion. Here, exclusion area changes caused by the pulse width increases are represented with an expanded sensing exclusion area 1045 (represented with a dotted line). A user may or may not be asked to accept the change before implementation.

FIG. 10E illustrates, by way of example, a user interface 1010D of the clinician programmer. As in FIG. 10A, this user interface shows a listing of parameters 1022 (with an increased frequency from parameters 1020) and calculation options 1031. Here, the sensing exclusion area 1046 represents an estimate of where sensing would not be possible, based on an active calculation of the stimulation waveform. The change of increasing the waveform frequency to 450 Hz (e.g., during a microburst) will reduce inter-pulse (i.e., inter-artifact) intervals. This causes exclusion areas 1047A, 1047B to emerge at the fringes of the electrical contacts, rather than only expanding from the CPS 1050. This demonstrates that as a general principle increasing waveform width expands an exclusion zone from a center point outwards. Whereas, as a general principle, increasing pulse rate causes an exclusion zone to expand from the edges.

FIG. 11 illustrates, by way of example, a data graph 1100, showing a relationship between a number of electrodes from stimulation to sensing (plotted on a vertical axis 1110) and a stimulation phase width (plotted on a horizontal axis 1120). Here, this graph shows that the size of a sensing area can be derived from collected data (e.g., collected in vivo) and mapped against stimulation parameters such as pulse width. The calculated or interpolated slope of the graph may inform the conduction velocity, which may be used to compute (via distance or time) or otherwise define (or help to define, or provide an alternative for) the exclusion zone and how the exclusion zone expands with increasing stimulation “width.”

Other variations may be provided. As an example, the y-axis on the graph may represent the number of SCS contacts (shown) based on implanted lead, the actual distance (e.g. in mm), or some other relative distance metric (such as vertebral level). The x-axis on the graph may represent the pulse width and is more deterministic, because pulse width is a setting. Further, the offset or axis may change depending on the waveform that is applied. For instance, the graph 1100 depicted in FIG. 11 assumes a rectangular biphasic waveform, also known as an “active recharge” method.

FIG. 12 illustrates, by way of example, an embodiment of an adaptive data processing flow to provide a neurostimulation treatment of a patient, which may be coordinated with the therapy and sensing programming objectives discussed above. Specifically, this data processing flow shows how a neurostimulation control system 1210 may implement aspects of closed-loop or partially-closed-loop feedback in therapy data processing functions 1212 and sensing data processing functions 1214, to provide improvements for therapy and sensing objectives from neurostimulation programming. Other user interfaces and functionality previously discussed in this document are not depicted for simplicity.

In this example, the user interface 710 (e.g., a patient user interface), is used to obtain an indication from a user (patient, caregiver, clinician or medical professional) related to therapy and sensing objectives, including input functionality 1204 related to identifying sensing and therapy objectives (further depicted in FIG. 7 and discussed above). The user interface 710 may provide information regarding the programming values or effects for therapy and sensing objectives, including output functionality 1202 for sensing capabilities (further depicted in FIGS. 8A-8C and 10A-10E and discussed above). The user interface 710 may use the input functionality 1204 and output functionality 1202 to assist a user via sensing area selection 1222 (e.g., to depict how to move a CPS to another anatomical area to improve the likelihood of collecting sensing data) and sensing exclusion area selection 1224 (e.g., to depict anatomical areas in which sensing data is unlikely to be collected). Such operations and functionality may involve other aspects of automated or manual programming adjustments discussed with reference to FIG. 9 or other examples above.

FIG. 12 also depicts the evaluation of device data 1230, such as sensor data 1232, therapy status data 1234, and other treatment aspects which may be obtained or derived from the stimulator 421, related neurostimulation programming values or settings, or external sensing devices. The device data 1230 and the information received with the user interface 710 (e.g., parameter values, sensing/therapy objectives) allow a patient state and device state to be determined within therapy data processing functions 1212 and sensing data processing functions 1214. Patient state and device state information may be evaluated by these functions 1212, 1214 to determine the efficacy of a therapy and/or the validity of sensing feedback from the therapy which can then cause adaptive changes to treatment and programming. The functions 1212, 1214 may separately or in combination control modulation effect calculations 1216 that drive changes to neurostimulation programming.

The remainder of the data processing flow illustrates how the patient state and device state—and related data from therapy and sensing feedback—can be used by the neurostimulation control system 1210. The neurostimulation control system 1210 may provide programming in a closed loop (or partially-closed-loop) system, to be adaptive to the patient state and device state, and to be adaptive to the therapy and sensing feedback. A programming system 1240 uses programming information 1242 provided from the neurostimulation control system 1210 as an input to program implementation logic 1250. The program implementation logic 1250 may be implemented by a parameter adjustment algorithm 1254, which affects a neurostimulation program selection 1252 or a neurostimulation program modification 1256. For instance, some parameter changes may be implemented by a simple modification to a program operation; other parameter changes may require a new program to be generated and/or deployed. The results of the parameter or program changes or selection provides various stimulation parameters 1270 to the stimulator 421, causing a different or new stimulation treatment effect 1260.

By way of example, operational parameters of the neurostimulation device which may be generated, identified, or evaluated by the neurostimulation control system 1210 may include amplitude, frequency, duration, pulse width, pulse type, patterns of neurostimulation pulses, waveforms in the patterns of pulses, and like settings with respect to the intensity, type, and location of neurostimulator output on individual or a plurality of respective leads and electrodes on the respective leads. The neurostimulator may use current or voltage sources to provide the neurostimulator output, and apply any number of control techniques to modify the electrical simulation applied to anatomical sites or systems related to pain or analgesic effect. In various embodiments, a neurostimulator program may be defined or updated to indicate parameters that define spatial, temporal, and informational characteristics for the delivery of modulated energy, including the definitions or parameters of pulses of modulated energy, waveforms of pulses, pulse blocks each including a burst of pulses, pulse trains each including a sequence of pulse blocks, train groups each including a sequence of pulse trains, and programs of such definitions or parameters, each including one or more train groups scheduled for delivery. Characteristics of the waveform that are defined in the program may include, but are not limited to the following: amplitude, pulse width, frequency, total charge injected per unit time, cycling (e.g., on/off time), pulse shape, number of phases, phase order, interphase time, charge balance, ramping, as well as spatial variance (e.g., electrode configuration changes over time). It will be understood that based on the many characteristics of the waveform itself, a program may have many parameter setting combinations that would be potentially available for use.

In still further examples, the approaches of closed-loop programming or human-responsive (i.e., partially-closed-loop) programming may be accompanied by various aspects of health monitoring, patient state tracking, treatment recommendation or reminders, and targeted health actions. The evaluation of therapy and sensing objectives in the neurostimulation control system 1210 may consider these and other aspects of patient activity and behavior. Patient state information also can be obtained from multiple data streams or data sources, including user inputs from patient devices (the user interface 710, apps on user phones, apps on other devices, multi-sensor watches, other user-input or user-tracking sensors).

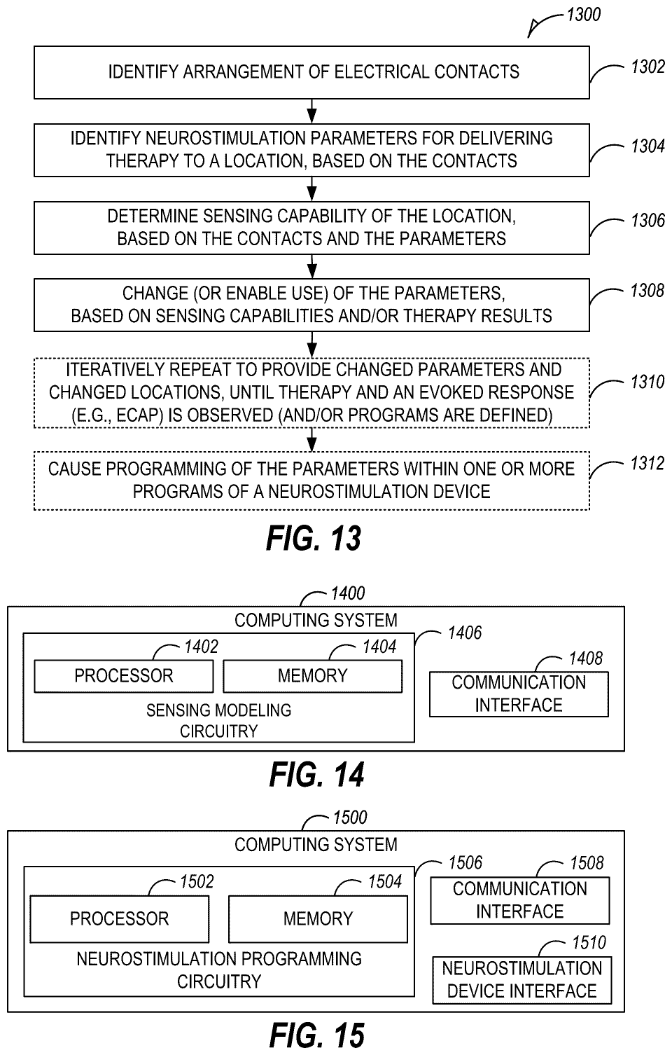

FIG. 13 illustrates, by way of example, an embodiment of a processing method 1300 implemented by a system or device to identify therapy and sensing capabilities in connection with a neurostimulation treatment. For example, the processing method 1300 can be embodied by electronic operations performed by one or more computing systems or devices (including those at a network-accessible remote service) that are specially programmed to implement the data analysis and/or neurostimulation data processing operations described herein. In specific examples, the operations of the method 1300 may be implemented through the systems and data flows depicted above in FIGS. 6 to 12, at a single entity or at multiple locations.

In an example, the method 1300 begins at operation 1302 by identifying an arrangement of electrical contacts of one or more implanted leads used or to be used for neurostimulation treatment. In some examples, this arrangement may include a first set of the electrical contacts to be used for delivering therapy, and a second set of the electrical contacts to be used for sensing an evoked response to the therapy. These arrangements may enable distinct stimulation configurations for therapeutic and/or sensing purposes. Thus, the same or different arrangements may be used for therapy and sensing purposes respectively. Further details of such arrangements are discussed in the examples above.

The method 1300 continues at operation 1304 by identifying neurostimulation parameters for delivering therapy (e.g., a neurostimulation treatment to be delivered to a patient with a plurality of electrical contacts of one or more implanted leads) to a neurostimulation location. This neurostimulation location may correspond to a CPS or other centralized point for the neurostimulation therapy results (e.g., a CPS provided with the first set of electrical contacts), although other locations may also be considered and evaluated. Consistent with the examples above, the neurostimulation parameters or values may relate to timing, amplitude, frequency, intensity, duration, pulse patterns, pulse shapes, a spatial location of pulses, waveform shapes, or a spatial location of waveform shapes, of modulated energy provided with one or more of the electrical contacts using at least one implanted lead. The identification of the neurostimulation parameters may be based on user interaction to set or change some attribute or value of the parameter, such as with a definition or a change to amplitude, pulse width, frequency, and percentage, of modulated energy (e.g., to be provided with a particular contact of the plurality of electrical contacts on the at least one implanted lead).

The method 1300 continues at operation 1306 by determining a sensing capability for sensing an evoked response to the therapy delivered (or to be delivered) to the neurostimulation location (e.g., using sensing capabilities provided using a second set of electrical contacts of the arrangement). In an example, this sensing capability is determined based on the arrangement of the electrodes and one or more of the neurostimulation parameters. In a further example, determining the sensing capabilities at the neurostimulation location includes determining whether an ECAP or another potential measurement is observable (or has been observed) from delivery of the therapy to the neurostimulator location.

In examples where the sensing capability is suitable (e.g., available for use) at the neurostimulation location, the method continues at operation 1308 to enable the use of the parameters. If the sensing capability is not suitable (or not available) at the identified location, then the method 1300 continues at operation 1310 to repeat changes until both of therapy and an evoked response are observed. In other examples, the method continues at operation 1310 until an acceptable or sufficient number of programs that generate either ECAPs or therapy are obtained (with the number of programs being pre-defined, or specified by a clinician or user). A user may also manually control additional iterations of the operations based on prior experience and/or training, and a user may also manually choose to not perform sensing at a particular location (for whatever reason) in response to stimulation-regardless of whether the particular location invokes an ECAP. The determination of a sensing capability may be based on the workflow for detecting ECAPs as depicted in FIG. 9, although other techniques may be used.

The method 1300 continues at operation 1308 by changing or enabling the use of the parameters, based on the sensing capabilities and/or the therapy results (e.g., enabling the use of the parameters based on determining that an evoked response is observed from the changed neurostimulation location and that the therapy results are also maintained at the changed neurostimulation location). In an example, this includes enabling use of changed neurostimulation parameters within one or more programs of a neurostimulation device, after determining that the sensing capability is available at the changed neurostimulation location. This may also include determining or receiving an indication (e.g., from the patient or a clinician) that the therapy remains effective for the patient at the changed neurostimulation location, and enabling use of the changed neurostimulation parameters within the one or more programs in response to this indication or determination. For instance, changing or enabling the parameters may occur in response to interaction received via a GUI, such as the clinical programming interfaces discussed with reference to FIGS. 7-8C or 10A-10D.

Additionally, in further examples, user interaction is received in a programming user interface (e.g., the programming interfaces depicted in FIGS. 7, 8A to 8C, and 10A to 10D) to change one or more neurostimulation parameters, and to display locations related to therapy outcomes, sensing capabilities, and arrangements of the electrical contacts. This programming user interface may receive input to change one or more values of neurostimulation parameters based on a suggested sensing area and/or a sensing exclusion area (based on the depictions of each area provided in the user interface). For instance, specific changes that are received in the programming user interface may include changes to amplitude, pulse width, frequency, and percentage, of modulated energy to be provided with a particular contact of the plurality of electrical contacts. Additionally, consistent with the examples above, a representation of a suggested sensing area may include a representation of a stimulation site that would produce the best results for sensing (e.g., based on the most contacts possible in a desired direction, as discussed with reference to FIGS. 8A to 8C above).

Operation 1310 includes optionally (and in some examples, iteratively) repeating the preceding operations (e.g., operations 1302, 1304, 1306, 1308) to produce changed neurostimulation parameters and changed neurostimulation locations, until an ECAP or other sensing measurement of an evoked response is observed at one or more of the changed neurostimulation locations. Any of the modeling and mapping techniques discussed with reference to FIGS. 8A to 11 may be integrated with these operations.

In further examples, the method 1300 continues at operation 1312 by optionally causing programming of the parameters within one or more programs of a neurostimulation device. Consequently, the programming of the neurostimulation device may be enabled for use or selection by the patient. The programming of the neurostimulation device may include use of other programming parameters or programs of the neurostimulation device, or other settings, consistent with the examples above.

FIG. 14 illustrates, by way of example, a block diagram of an embodiment of a system 1400 (e.g., a computing system) for performing patient data analysis in connection with the sensing and therapy objectives discussed above. The system 1400 may be integrated with or coupled to a computing device, a remote control device, patient programmer device, clinician programmer device, program modeling system, or other external device, deployed with neurostimulation treatment. In some examples, the system 1400 may be a networked device (server) connected via a network (or combination of networks) which communicates to one or more devices (clients) using a communication interface 1408 (e.g., communication hardware which implements software network interfaces and services). The network may include local, short-range, or long-range networks, such as Bluetooth, cellular, IEEE 802.11 (Wi-Fi), or other wired or wireless networks.

The system 1400 includes a processor 1402 and a memory 1404, which can be optionally included as part of sensing modeling circuitry 1406. The processor 1402 may be any single processor or group of processors that act cooperatively. The memory 1404 may be any type of memory, including volatile or non-volatile memory. The memory 1404 may include instructions, which when executed by the processor 1402, cause the processor 1402 to determine programming parameter changes that enable improved sensing capabilities, or to determine other sensing effects or outcomes via the sensing modeling circuitry 1406. Thus, electronic operations in the system 1400 may be performed by the processor 1402 or the circuitry 1406.

For example, the processor 1402 or circuitry 1406 may implement any of the features of the method 1300 (such as operations 1302-1310) to evaluate and identify neurostimulation programming parameters, to determine modified programming values, to identify and evaluate anatomical areas and therapy outcomes, to model sensing outcomes, and to prompt and observe for sensing capabilities based on the testing or implementation of programming values. It will be understood that the processor 1402 or circuitry 1406 may also implement or control aspects of the logic and processing described above with reference to FIGS. 6-13, for use in a various forms of closed-loop or open-loop device programming or related device actions.

FIG. 15 illustrates, by way of example, a block diagram of an embodiment of a system 1500 (e.g., a computing system) implementing neurostimulation programming circuitry 1506 to cause programming of an implantable electrical neurostimulation device, for accomplishing the therapy and sensing programming objectives in a human subject as discussed herein. The system 1500 may be operated by a clinician, a patient, a caregiver, a medical facility, a research institution, a medical device manufacturer or distributor, and embodied in a number of different computing platforms. The system 1500 may be a remote control device, patient programmer device, program modeling system, or other external device, including a regulated device used to directly implement programming commands and modification with a neurostimulation device. In some examples, the system 1500 may be a networked device connected via a network (or combination of networks) to a computing system operating a user interface computing system using a communication interface 1508. The network may include local, short-range, or long-range networks, such as Bluetooth, cellular, IEEE 802.11 (Wi-Fi), or other wired or wireless networks.

The system 1500 includes a processor 1502 and a memory 1504, which can be optionally included as part of neurostimulation programming circuitry 1506. The processor 1502 may be any single processor or group of processors that act cooperatively. The memory 1504 may be any type of memory, including volatile or non-volatile memory. The memory 1504 may include instructions, which when executed by the processor 1502, cause the processor 1502 to implement the features of the neurostimulation programming circuitry 1506. Thus, the electronic operations in the system 1500 may be performed by the processor 1502 or the circuitry 1506.

The processor 1502 or circuitry 1506 may directly or indirectly implement neurostimulation operations associated with the method 1300, including to change or enable the use of parameters (operation 1310) or to cause programming of the parameters within one or more programs of the neurostimulator (operation 1312). The processor 1502 or circuitry 1506 may further provide data and commands to assist the processing and implementation of the programming using communication interface 1508. It will be understood that the processor 1502 or circuitry 1506 may also implement other aspects of the device data processing or device programming functionality described above with reference to FIGS. 6-13.

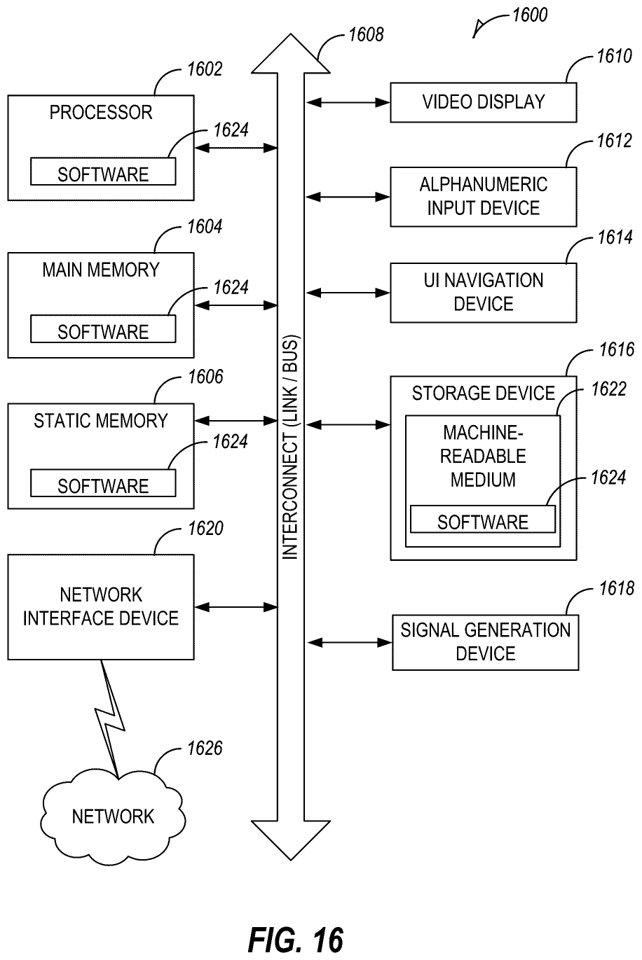

FIG. 16 is a block diagram illustrating a machine in the example form of a computer system 1600, within which a set or sequence of instructions may be executed to cause the machine to perform any one of the methodologies discussed herein, according to an example embodiment. In alternative embodiments, the machine operates as a standalone device or may be connected (e.g., networked) to other machines. In a networked deployment, the machine may operate in the capacity of either a server or a client machine in server-client network environments, or it may act as a peer machine in peer-to-peer (or distributed) network environments. The machine may be a personal computer (PC), a tablet PC, a hybrid tablet, a personal digital assistant (PDA), a mobile telephone, an implantable pulse generator (IPG), an external remote control (RC), a User's Programmer (CP), or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. Similarly, the term “processor-based system” shall be taken to include any set of one or more machines that are controlled by or operated by a processor (e.g., a computer) to individually or jointly execute instructions to perform any one or more of the methodologies discussed herein.

Example computer system 1600 includes at least one processor 1602 (e.g., a central processing unit (CPU), a graphics processing unit (GPU) or both, processor cores, compute nodes, etc.), a main memory 1604 and a static memory 1606, which communicate with each other via a link 1608 (e.g., bus). The computer system 1600 may further include a video display unit 1610, an alphanumeric input device 1612 (e.g., a keyboard), and a user interface (UI) navigation device 1614 (e.g., a mouse). In one embodiment, the video display unit 1610, input device 1612 and UI navigation device 1614 are incorporated into a touch screen display. The computer system 1600 may additionally include a storage device 1616 (e.g., a drive unit), a signal generation device 1618 (e.g., a speaker), a network interface device 1620, and one or more sensors (not shown), such as a global positioning system (GPS) sensor, compass, accelerometer, or other sensor. It will be understood that other forms of machines or apparatuses (such as PIG, RC, CP devices, and the like) that are capable of implementing the methodologies discussed in this disclosure may not incorporate or utilize every component depicted in FIG. 16 (such as a GPU, video display unit, keyboard, etc.).

The storage device 1616 includes a machine-readable medium 1622 on which is stored one or more sets of data structures and instructions 1624 (e.g., software) embodying or utilized by any one or more of the methodologies or functions described herein. The instructions 1624 may also reside, completely or at least partially, within the main memory 1604, static memory 1606, and/or within the processor 1602 during execution thereof by the computer system 1600, with the main memory 1604, static memory 1606, and the processor 1602 also constituting machine-readable media.

While the machine-readable medium 1622 is illustrated in an example embodiment to be a single medium, the term “machine-readable medium” may include a single medium or multiple media (e.g., a centralized or distributed database, and/or associated caches and servers) that store the one or more instructions 1624. The term “machine-readable medium” shall also be taken to include any tangible (e.g., non-transitory) medium that is capable of storing, encoding or carrying instructions for execution by the machine and that cause the machine to perform any one or more of the methodologies of the present disclosure or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including but not limited to, by way of example, semiconductor memory devices (e.g., electrically programmable read-only memory (EPROM), electrically erasable programmable read-only memory (EEPROM)) and flash memory devices; magnetic disks such as internal hard disks and removable disks; magneto-optical disks; and CD-ROM and DVD-ROM disks.

The instructions 1624 may further be transmitted or received over a communications network 1626 using a transmission medium via the network interface device 1620 utilizing any one of a number of well-known transfer protocols (e.g., HTTP). Examples of communication networks include a local area network (LAN), a wide area network (WAN), the Internet, mobile telephone networks, plain old telephone (POTS) networks, and wireless data networks (e.g., Wi-Fi, 3G, and 4G LTE/LTE-A or 5G networks). The term “transmission medium” shall be taken to include any intangible medium that is capable of storing, encoding, or carrying instructions for execution by the machine, and includes digital or analog communications signals or other intangible medium to facilitate communication of such software.

The above detailed description is intended to be illustrative, and not restrictive. The scope of the disclosure should, therefore, be determined with references to the appended claims, along with the full scope of equivalents to which such claims are entitled.

What is claimed is:

1. A device to identify therapy and sensing capabilities of neurostimulation programming, the device comprising:
one or more processors; and
one or more memory devices comprising instructions, which when executed by the one or more processors, cause the one or more processors to:
identify an arrangement for a plurality of electrical contacts of one or more implanted leads;
identify neurostimulation parameters for delivering therapy to a neurostimulation location using a first set of electrical contacts of the arrangement;
determine a sensing capability for sensing an evoked response to the therapy delivered to the neurostimulation location, using a second set of electrical contacts of the arrangement, wherein the sensing capability is determined based on the arrangement and one or more of the neurostimulation parameters; and
change at least a portion of the neurostimulation parameters for delivering the therapy, in response to a determination that the sensing capability is not available at the neurostimulation location, to provide changed neurostimulation parameters for delivering therapy to a changed neurostimulation location.

2. The device of claim 1, wherein the neurostimulation location corresponds to a central point of stimulation provided with the first set of electrical contacts, and wherein to determine the sensing capability at the neurostimulation location includes to determine whether an evoked compound action potential (ECAP) is observable.

3. The device of claim 1, wherein operations to identify the neurostimulation parameters, to determine the sensing capability, and to change the neurostimulation parameters, are iteratively repeated to produce the changed neurostimulation parameters associated with changed neurostimulation locations, until an evoked compound action potential (ECAP) is observed from use of the changed neurostimulation parameters at one of the changed neurostimulation locations.

4. The device of claim 1, wherein the instructions further cause the one or more processors to perform operations that:
determine that the sensing capability is available at the changed neurostimulation location; and
enable use of the changed neurostimulation parameters within one or more programs of a neurostimulation device.

5. The device of claim 4, wherein the instructions further cause the one or more processors to perform operations that:
receive an indication that the therapy remains effective for a patient at the changed neurostimulation location;
wherein the changed neurostimulation parameters are enabled for use within the one or more programs in response to the indication that the therapy remains effective for the patient.

6. The device of claim 5, wherein the instructions further cause the one or more processors to perform operations that:
determine that the sensing capability is available via the second set of electrical contacts of the arrangement, based on use of the changed neurostimulation parameters;
wherein the changed neurostimulation parameters are enabled for use within the one or more programs in response to a determination that both the sensing capability remains available and the therapy remains effective for the patient.

7. The device of claim 6, wherein the change to the neurostimulation parameters is based on a suggested sensing area or a sensing exclusion area;
wherein a size and a location of the suggested sensing area or the sensing exclusion area is based on lead geometry and stimulation waveform characteristics.

8. The device of claim 1, wherein the instructions further cause the one or more processors to:
display, in a programming user interface, a representation of the neurostimulation location and a representation of the plurality of electrical contacts;
wherein the change to the neurostimulation parameters is based on settings of the neurostimulation parameters that are modifiable in the programming user interface.

9. The device of claim 8, wherein the instructions further cause the one or more processors to:
display, in the programming user interface, a representation of a suggested sensing area, relative to the area of the therapy;
display, in the programming user interface, a representation of a sensing exclusion area, relative to the area of the therapy; and
receive user interaction in the programming user interface to change at least one value of the neurostimulation parameters or the neurostimulation location, based on the suggested sensing area or the sensing exclusion area.

10. The device of claim 9, wherein the user interaction to change the at least one value of the neurostimulation parameters relates to a change to amplitude, pulse width, frequency, and percentage, of modulated energy to be provided with a particular contact of the plurality of electrical contacts.

11. A method for identifying therapy and sensing capabilities of neurostimulation programming, comprising:
identifying an arrangement for a plurality of electrical contacts of one or more implanted leads;
identifying neurostimulation parameters for delivering therapy to a neurostimulation location using a first set of electrical contacts of the arrangement;
determining a sensing capability for sensing an evoked response to the therapy delivered to the neurostimulation location, using a second set of electrical contacts of the arrangement, wherein the sensing capability is determined based on the arrangement and one or more of the neurostimulation parameters; and
changing at least a portion of the neurostimulation parameters for delivering the therapy, in response to a determination that the sensing capability is not available at the neurostimulation location, to provide changed neurostimulation parameters for delivering therapy to a changed neurostimulation location.

12. The method of claim 11, wherein the neurostimulation location corresponds to a central point of stimulation provided with the first set of electrical contacts, and wherein determining the sensing capability at the neurostimulation location includes determining whether an evoked compound action potential (ECAP) is observable.

13. The method of claim 11, wherein identifying the neurostimulation parameters, determining the sensing capability, and changing the neurostimulation parameters, are iteratively repeated to produce the changed neurostimulation parameters associated with changed neurostimulation locations, until an evoked compound action potential (ECAP) is observed from use of the changed neurostimulation parameters at one of the changed neurostimulation locations.

14. The method of claim 11, further comprising:
determining that the sensing capability is available at the changed neurostimulation location; and
enabling use of the changed neurostimulation parameters within one or more programs of a neurostimulation device.

15. The method of claim 14, further comprising:
receiving an indication that the therapy remains effective for a patient at the changed neurostimulation location;
wherein the changed neurostimulation parameters are enabled for use within the one or more programs in response to the indication that the therapy remains effective for the patient.

16. The method of claim 15, further comprising:
determining that the sensing capability is available via the second set of electrical contacts of the arrangement, based on use of the changed neurostimulation parameters;
wherein the changed neurostimulation parameters are enabled for use within the one or more programs in response to determining that both the sensing capability remains available and the therapy remains effective for the patient.

17. The method of claim 16, wherein the change to the neurostimulation parameters is based on a suggested sensing area or a sensing exclusion area;
wherein a size and a location of the suggested sensing area or the sensing exclusion area is based on lead geometry and stimulation waveform characteristics.

18. The method of claim 11, further comprising:
displaying, in a programming user interface, a representation of the neurostimulation location and a representation of the plurality of electrical contacts;
wherein the change to the neurostimulation parameters is based on settings of the neurostimulation parameters that are modifiable in the programming user interface.

19. The method of claim 18, further comprising:
displaying, in the programming user interface, a representation of a suggested sensing area, relative to the area of the therapy;
displaying, in the programming user interface, a representation of a sensing exclusion area, relative to the area of the therapy; and
receive user interaction in the programming user interface to change at least one value of the neurostimulation parameters or the neurostimulation location, based on the suggested sensing area or the sensing exclusion area.

20. The method of claim 19, wherein the user interaction to change the at least one value of the neurostimulation parameters relates to a change to amplitude, pulse width, frequency, and percentage, of modulated energy to be provided with a particular contact of the plurality of electrical contacts.
